(19) United States
(12) Patent Application Publication
(10) Pub. No.: US 2017/0007135 A1
(43) Pub. Date: Jan. 12, 2017

(54) BLOOD VESSEL INDEX VALUE CALCULATION APPARATUS, BLOOD VESSEL INDEX VALUE CALCULATION METHOD, AND BLOOD VESSEL INDEX VALUE CALCULATION PROGRAM

(71) Applicant: OMRON HEALTHCARE Co., Ltd., Kyoto (JP)

(72) Inventors: Miki IMAMURA, Kyoto (JP); Toshihiko OGURA, Kyoto (JP); Yukiya SAWANOI, Kyoto (JP); Hironori SATO, Kyoto (JP)

(73) Assignee: OMRON HEALTHCARE Co., Ltd., Kyoto (JP)

(21) Appl. No.: 15/272,480

(22) Filed: Sep. 22, 2016

Related U.S. Application Data
(63) Continuation of application No. PCT/JP2015/054235, filed on Feb. 17, 2015.

(30) Foreign Application Priority Data
Mar. 24, 2014 (JP) 2014-060267

(51) Int. Cl. A61B 5/021; A61B 8/02; A61B 5/00; A61B 5/02; A61B 5/024

(52) U.S. Cl. CPC A61B 5/02108; A61B 5/02007; A61B 5/02422; A61B 5/6824; A61B 8/02

(57) ABSTRACT

Provided is a frequency transfer characteristic blood vessel index value calculation apparatus including a pulse wave acquisition unit that acquires first pulse wave data of a pulse wave at a first site, and second pulse wave data of a pulse wave at a second site; a pulse wave frequency characteristic derivation unit that derives a first frequency characteristic of the first pulse wave from the first pulse wave data and derives a second frequency characteristic of the second pulse wave from the second pulse wave data; a frequency transfer characteristic calculation unit that calculates a frequency transfer characteristic for a system that uses the first pulse wave as input and the second pulse wave as output; a frequency transfer characteristic correction unit that corrects the frequency transfer characteristic.

### TECHNICAL FIELD

The present invention relates to a blood vessel index value calculation apparatus and a blood vessel index value calculation method for calculating an index value indicating the state of a blood vessel based on information on a pulse wave acquired from a measurement subject.

The present invention relates to a blood vessel index value calculation program for causing a computer to execute a method of calculating an index value indicating the state of a blood vessel based on information on a pulse wave acquired from a measurement subject.

### BACKGROUND ART

Measurement of an ankle brachial (pressure) index (ABI) is extremely important in that it provides a reliable and objective index for diagnosis of peripheral arterial disease (PAD) or arteriosclerosis obliterans (ASO). The ankle brachial (pressure) index (hereinafter referred to as “ABI” as well) is defined as a value obtained by dividing the ankle blood pressures of a measurement subject by the upper-arm blood pressures. The ankle blood pressures in this context are the blood pressures (systolic blood pressures) of the posterior tibial arteries (PT) or the blood pressures of the dorsalis pedis arteries (DP) of the left and right feet, and ordinarily, the higher blood pressure value is used as the ankle blood pressure. On the other hand, as for the upper-arm blood pressure, the higher value of the left and right upper-arm blood pressures (systolic blood pressures) is used as the upper-arm blood pressure. Accordingly, ordinarily, calculation of ABI requires measurement of the systolic blood pressure at an upper arm and an ankle of the measurement subject.

Patent Literature 1 (JP 2013-094262A) discloses a measurement apparatus that calculates an index value (hereinafter referred to as an “ABI estimate value”) corresponding to the ABI based on a pulse wave of the measurement subject. With this measurement apparatus, an index indicating the sharpness of the pulse wave, an index indicating a rising characteristic value of the ankle pulse wave, a pulse amplitude, an index indicating a transfer function for a pulse wave from an upper extremity to a lower extremity (such as “upper area”, “ratio of upper area to lower area”, and “segment maximum” of a step response), and the like are calculated based on pulse wave signals of the upper extremity and lower extremity acquired from the measurement subject, and based on these values, an ABI estimate value is calculated (paragraphs [0054] to [0069] and FIGS. 22, 23 to 27, etc. of Patent Literature 1). The ABI estimate value calculated in this manner indicates a coefficient of determination (contribution ratio) of 0.663 with respect to a true ABI value (the value of the ABI obtained by actually measuring the systolic blood pressure at the upper arm and ankle of the measurement subject, hereinafter referred to as “ABI measurement value”) (FIG. 27 in Patent Literature 1).

### CITATION LIST

### Patent Literature

Patent Literature 1: JP 2013-094262A

### SUMMARY OF INVENTION

### Technical Problem

However, even higher accuracy is desired in the index value indicating the state of a blood vessel, such as the ABI estimate value. For example, it is desired that the above-described index value such as the ABI estimate value has a higher correlation with the ABI measurement value. In view of this, the present invention aims to provide a blood vessel index value calculation apparatus and a blood vessel index value calculation method according to which it is possible to calculate an index value indicating the state of a blood vessel with higher accuracy than with prior techniques, based on information on a pulse wave obtained from a measurement subject.

The present invention also aims to provide a blood vessel index value calculation program according to which it is possible to calculate an index value indicating the state of a blood vessel with higher accuracy than with prior techniques, based on information on a pulse wave obtained from a measurement subject.

### Solution to Problem

In order to solve the foregoing problems, a blood vessel index value calculation apparatus that calculates an index value indicating a state of a blood vessel of a measurement subject according to an embodiment of the present invention includes: a pulse wave acquisition unit configured to acquire first pulse wave data that includes time series information on a first pulse wave, which is a pulse wave at a first measurement site of the measurement subject, and second pulse wave data that includes time series information on a second pulse wave, which is a pulse wave at a second measurement site of the measurement subject; a pulse wave frequency characteristic derivation unit configured to derive a first frequency characteristic, which is a frequency characteristic of the first pulse wave, by converting the acquired first pulse wave data into a frequency space, and to derive a second frequency characteristic, which is a frequency characteristic of the second pulse wave, by converting the acquired second pulse wave data into a frequency space; a frequency transfer characteristic calculation unit configured to, based on the first frequency characteristic and the second frequency characteristic, calculate a frequency transfer characteristic for a vascular system that includes the blood vessel and uses the first pulse wave as input and the second pulse wave as output; a frequency transfer characteristic correction unit configured to correct the calculated frequency transfer characteristic; a response calculation unit configured to calculate a response of the vascular system to pre-determined referential input using the corrected frequency transfer characteristic; and an index value calculation unit configured to calculate an index value indicating the state of the blood vessel based on the calculated response. The frequency transfer characteristic correction unit corrects a frequency gain characteristic of the frequency transfer characteristic by weighting the frequency gain characteristic based on a frequency amplitude characteristic of the first frequency characteristic, and corrects the frequency transfer characteristic based on the corrected frequency gain characteristic.

With the blood vessel index value calculation apparatus according to the embodiment of the present invention, the pulse wave acquisition unit acquires time series information (first and second pulse wave data) on the pulse waves of the first and second measurement sites of the measurement subject, the pulse wave frequency characteristic derivation unit derives frequency characteristics (first and second frequency characteristics) of the pulse waves of the first and second measurement sites, and the frequency transfer characteristic calculation unit calculates the frequency transfer characteristic of the vascular system using the first and second frequency characteristics. Then, the frequency transfer characteristic correction unit corrects the frequency gain characteristic by weighting the frequency gain characteristic of the calculated frequency transfer characteristic based on the frequency amplitude characteristic of the first frequency characteristic and corrects the frequency transfer characteristic based on the corrected frequency gain characteristic. Finally, the response calculation unit calculates a response of the vascular system to the referential input using the thus-corrected frequency transfer characteristic, and the index value calculation unit calculates the index value indicating the state of the blood vessel based on the response calculated by the response calculation unit.

Originally, the frequency gain characteristic of the frequency transfer characteristic is the ratio between the input and output of each frequency component and is not directly related to the relative magnitude relationship between the frequency components included in the input. On the other hand, the pulse wave includes the component of the frequency (frequency of fundamental wave) that coincides with the reciprocal of the pulse rate of the measurement subject and the components of the harmonic waves, and the amplitudes of the components decrease exponentially in the direction from the fundamental wave to the harmonic waves. In view of this, in the present embodiment, the frequency gain characteristic of the frequency transfer characteristic is weighted based on the frequency amplitude characteristic of the first frequency characteristic such that the response characteristics of the components of the fundamental wave and comparatively low-order harmonic waves, which are included in the pulse wave, are emphasized in comparison to the response characteristics of the components of the comparatively high-order harmonic waves. Specifically, the frequency gain characteristic is weighted in accordance with the proportion of the peak of the frequency of the fundamental wave in an amplitude spectrum that has been converted into a frequency space, and the peaks of one or more harmonic waves. The response calculation unit calculates the response of the vascular system to the referential input using the thus-corrected frequency transfer characteristic, and the index value calculation unit calculates the index value indicating the state of the blood vessel based on the calculated response. By doing so, in the present embodiment, in the response to the referential input, the contributions from the components of the fundamental wave and the comparatively low-order harmonic waves of the pulse wave are emphasized, and the index value calculation unit can accurately calculate the index value by calculating the index value indicating the state of the blood vessel using such a response.

In order to solve the foregoing problems, a blood vessel index value calculation apparatus according to an embodiment of the present invention is such that based on the first frequency characteristic, the frequency transfer characteristic correction unit corrects the frequency gain characteristic of the frequency transfer characteristic such that frequency gain characteristics of a first frequency, which is equivalent to a frequency of a fundamental wave of the frequency transfer characteristic, and a second frequency, which is equivalent to a frequency of a second harmonic wave of the first frequency, pass through gains at the first frequency and the second frequency and change linearly, corrects a frequency phase characteristic of the frequency transfer characteristic such that frequency phase characteristics of the first frequency and the second frequency pass through phases at the first frequency and the second frequency and change linearly, and corrects the frequency transfer characteristic based on the corrected frequency gain characteristic and the corrected frequency phase characteristic.

With the blood vessel index value calculation apparatus according to the embodiment of the present invention, the frequency transfer characteristic is corrected such that the first and second frequencies, which are respectively equal to the frequency of the fundamental wave and the frequency of the second harmonic wave of the pulse wave, are connected linearly in the frequency gain characteristic and the frequency phase characteristic of the frequency transfer characteristic. By doing so, it is possible to suppress the contributions to the response to the referential input of the components of the frequencies thought to not originate in the pulse wave among the components of the frequencies in the range between the first frequency and the second frequency in the frequency transfer characteristic, and it is possible for the later-described index value calculation unit to accurately calculate the index value (ABWI value) indicating the state of the blood vessel.

In order to solve the foregoing problems, a blood vessel index value calculation apparatus according to an embodiment of the present invention is such that based on the first frequency characteristic, the frequency transfer characteristic correction unit corrects the frequency gain characteristic of the frequency transfer characteristic such that the frequency gain characteristics of the second frequency and a third frequency, which is equivalent to a frequency of a third harmonic wave of the first pulse wave, pass through gains at the second frequency and the third frequency and change linearly, corrects the frequency phase characteristic of the frequency transfer characteristic such that the frequency phase characteristics of the second frequency and the third frequency pass through phases at the second frequency and the third frequency and change linearly, and corrects the frequency transfer characteristic based on the corrected frequency gain characteristic and the corrected frequency phase characteristic.

With the blood vessel index value calculation apparatus according to the embodiment of the present invention, the frequency transfer characteristic is corrected such that the second and third frequencies, which are respectively equal to the frequency of the second harmonic wave and the frequency of the third harmonic wave of the pulse wave, are connected linearly in the frequency gain characteristic and the frequency phase characteristic of the frequency transfer characteristic. By doing so, it is possible to suppress the contributions to the response to the referential input of the components of the frequencies thought to not originate in the pulse wave among the components of the frequencies in the range between the second frequency and the third frequency in the frequency transfer characteristic, and it is possible for the later-described index value calculation unit to accurately calculate the index value (ABWI value) indicating the state of the blood vessel.

In order to solve the foregoing problems, a blood vessel index value calculation apparatus according to an embodiment of the present invention is such that the frequency transfer characteristic correction unit corrects the frequency transfer characteristic by restricting a frequency band of the frequency transfer characteristic to be within a range from a frequency lower than the frequency of the fundamental wave of the first pulse wave to 10 Hertz. Here, a frequency lower than the frequency of the fundamental wave of the first pulse wave is a frequency equal to a value obtained by subtracting at least the frequency resolution of the pulse wave frequency characteristic derivation unit from the frequency of the fundamental wave of the first pulse wave, for example.

With the blood vessel index value calculation apparatus according to the embodiment of the present invention, the influence of the components of the comparatively high-order harmonic waves of the pulse wave on the response is removed by limiting the frequency band of the frequency transfer characteristic to 10 Hertz or less. By doing so, the contributions from the components of the comparatively high-order harmonic waves of the pulse wave are removed (or at least reduced) in the response to the referential input, and the index value calculation unit can accurately calculate the index value by using such a response to calculate the index value indicating the state of the blood vessel.

In order to solve the foregoing problems, a blood vessel index value calculation apparatus according to an embodiment of the present invention is such that the referential input is in the form of a step function, the response calculation unit calculates the response of the vascular system to the referential input, and the index value calculation unit calculates the index value indicating the state of the blood vessel based on an amount of time that elapses before a maximum first appears in the response.

With the blood vessel index value calculation apparatus according to the embodiment of the present invention, the index value indicating the state of the blood vessel is calculated based on the amount of time that elapses before a maximum first appears in the response. By doing so, the index value indicating the state of the blood vessel is calculated accurately.

In order to solve the foregoing problems, a blood vessel index value calculation apparatus according to an embodiment of the present invention is such that based on the first frequency characteristic, the frequency transfer characteristic correction unit corrects the frequency phase characteristic of the frequency transfer characteristic such that the frequency phase characteristic in a frequency range of being less than or equal to the first frequency reaches a value equal to the phase at the first frequency, and corrects the frequency transfer characteristic based on the corrected frequency phase characteristic.

In order to solve the foregoing problems, a blood vessel index value calculation apparatus according to an embodiment of the present invention is such that based on the first frequency characteristic, the frequency transfer characteristic correction unit corrects the frequency gain characteristic of the frequency transfer characteristic such that the frequency gain characteristic in a frequency range of being less than or equal to the first frequency, which is equivalent to the frequency of the fundamental wave of the first pulse wave, reaches a value equal to the gain at the first frequency, and corrects the frequency transfer characteristic based on the corrected frequency gain characteristic.

The blood vessel index value calculation apparatus according to the embodiment of the present invention corrects the frequency transfer characteristic such that in the frequency range of being less than or equal to the first frequency, which corresponds to the reciprocal of the pulse rate, at least one of the frequency gain characteristic and the frequency phase characteristic is the same value as the value of the first frequency. By doing so, the influence of the magnitude of the pulse rate of the measurement subject on the response can be reduced, and the index value indicating the state of the blood vessel is accurately calculated.

In order to solve the foregoing problems, a blood vessel index value calculation apparatus according to an embodiment of the present invention is such that the index value calculation unit calculates the index value indicating the state of the blood vessel based on the response and the frequency of the fundamental wave of the first pulse wave.

With the blood vessel index value calculation apparatus according to the embodiment of the present invention, the index value indicating the state of the blood vessel is calculated based on the response and the frequency of the fundamental wave of the first pulse wave, or in other words, the pulse rate (the reciprocal of the pulse rate) of the measurement subject. Specifically, a linear bond between a characteristic amount of the response and the measurement subject is obtained by using a constant coefficient obtained in advance by regression analysis, and this is used as the index value. By doing so, the influence of the magnitude of the pulse rate of the measurement subject on the response can be reduced, and the index value indicating the state of the blood vessel is accurately calculated.

In order to solve the foregoing problems, a blood vessel index value calculation apparatus according to an embodiment of the present invention is such that the pulse wave frequency characteristic derivation unit divides the first pulse wave data and the second pulse wave data into a plurality of data frames, derives frequency characteristics of the data frames of at least one of the first pulse wave data and the second pulse wave data, obtains the lowest frequency exhibiting a peak in each derived frequency characteristic, specifies and excludes a data frame including noise based on the obtained lowest frequency, and derives the first frequency characteristic and the second frequency characteristic based on the data frames of at least one of the first pulse wave data and the second pulse wave data that were not excluded, and on the corresponding data frames of at least the other of the first pulse wave data and the second pulse wave data.

The blood vessel index value calculation apparatus according to the embodiment of the present invention determines whether or not noise is included in each data frame, and if it is determined that noise is included in a data frame, that data frame is excluded (rejected). By doing so, the influence of noise that is added during pulse wave data acquisition is reduced, and the index value indicating the state of the blood vessel is calculated accurately.

In order to solve the foregoing problems, a blood vessel index value calculation method for calculating an index value indicating a state of a blood vessel of a measurement subject according to another embodiment of the present invention includes: a step in which an arithmetic unit of the blood vessel index value calculation apparatus acquires first pulse wave data that includes time series information on a first pulse wave, which is a pulse wave at a first measurement site of the measurement subject, and second pulse wave data that includes time series information on a second pulse wave, which is a pulse wave at a second measurement site of the measurement subject; a step in which the arithmetic unit derives a first frequency characteristic, which is a frequency characteristic of the first pulse wave, by converting the acquired first pulse wave data into a frequency space, and derives a second frequency characteristic, which is a frequency characteristic of the second pulse wave, by converting the acquired second pulse wave data into a frequency space; a step in which the arithmetic unit, based on the first frequency characteristic and the second frequency characteristic, calculates a frequency transfer characteristic for a vascular system that includes the blood vessel and uses the first pulse wave as input and the second pulse wave as output; a step in which the arithmetic unit corrects the frequency transfer characteristic calculated in the step of calculation; a step in which the arithmetic unit calculates a response of the blood vessel system to pre-determined referential input using the frequency transfer characteristic corrected in the step of correction; and a step in which the arithmetic unit calculates an index value indicating the state of the blood vessel based on the response calculated in the step of calculating the response. The step of correction includes a step in which the arithmetic unit corrects a frequency gain characteristic of the frequency transfer characteristic by weighting the frequency gain characteristic based on a frequency amplitude characteristic of the first frequency characteristic, and corrects the frequency transfer characteristic based on the corrected frequency gain characteristic.

With the blood vessel index value calculation method according to the other embodiment of the present invention, the frequency characteristics (first and second frequency characteristics) of the pulse wave of the first and second measurement sites are derived using the acquired first and second pulse wave data, and the frequency transfer characteristic of the vascular system is calculated based on the first and second frequency characteristics. Then, the frequency gain characteristic is corrected due to the frequency gain characteristic of the calculated frequency transfer characteristic being weighted based on the frequency amplitude characteristic of the first frequency characteristic, and the frequency transfer characteristic is corrected based on the corrected frequency gain characteristic. Finally, the response of the vascular system to the referential input is calculated using the thus-corrected frequency transfer characteristic, and the index value indicating the state of the blood vessel is calculated based on the response.

In the present embodiment, the contribution from the components of the fundamental wave and the comparatively low-order harmonic waves of the pulse wave are emphasized in the response to the referential input, and by calculating the index value indicating the state of the blood vessel using such a response, the index value is calculated accurately.

In order to solve the foregoing problems, a blood vessel index value calculation program for causing a computer to execute a method for calculating an index value indicating a state of a blood vessel of a measurement subject according to another embodiment of the present invention is a blood vessel index value calculation program for causing a computer to execute a method for calculating an index value indicating a state of a blood vessel of a measurement subject, the method including: a step of acquiring first pulse wave data that includes time series information on a first pulse wave, which is a pulse wave at a first measurement site of the measurement subject, and second pulse wave data that includes time series information on a second pulse wave, which is a pulse wave at a second measurement site of the measurement subject; a step of deriving a first frequency characteristic, which is a frequency characteristic of the first pulse wave, based on the acquired first pulse wave data, and deriving a second frequency characteristic, which is a frequency characteristic of the second pulse wave, based on the acquired second pulse wave data; a step of, based on the first frequency characteristic and the second frequency characteristic, calculating a frequency transfer characteristic for a vascular system that includes the blood vessel and uses the first pulse wave as input and the second pulse wave as output; a step of correcting the frequency transfer characteristic calculated in the step of calculation; a step of calculating a response of the blood vessel system to pre-determined referential input using the frequency transfer characteristic corrected in the step of correction; and a step of calculating an index value indicating the state of the blood vessel based on the response calculated in the step of calculating the response. The step of correction includes a step of correcting a frequency gain characteristic of the frequency transfer characteristic by weighting the frequency gain characteristic based on a frequency amplitude characteristic of the first frequency characteristic, and correcting the frequency transfer characteristic based on the corrected frequency gain characteristic.

With the blood vessel index value calculation program according to the other embodiment of the present invention, the frequency characteristics (first and second frequency characteristics) of the pulse wave of the first and second measurement sites are derived using the acquired first and second pulse wave data, and the frequency transfer characteristic of the vascular system is calculated based on the first and second frequency characteristics. Then, the frequency gain characteristic is corrected due to the frequency gain characteristic of the calculated frequency transfer characteristic being weighted based on the frequency amplitude characteristic of the first frequency characteristic, and the frequency transfer characteristic is corrected based on the corrected frequency gain characteristic. Finally, the response of the vascular system to the referential input is calculated using the thus-corrected frequency transfer characteristic, and the index value indicating the state of the blood vessel is calculated based on the response.

In the present embodiment, the contributions from the components of the fundamental wave and the comparatively low-order harmonic waves of the pulse wave are emphasized in the response to the referential input, and by calculating the index value indicating the state of the blood vessel using such a response, the index value is calculated accurately.

In the present specification, the frequency transfer characteristic of a system includes at least one or both of the frequency gain characteristic and the frequency phase characteristic of that system. The frequency transfer characteristic of a system is represented by the transfer function of that system, for example.

In the present specification, the frequency characteristic of a piece of time series data includes at least one or both of the frequency amplitude characteristic and the frequency phase characteristic of that data. The frequency characteristic of a piece of time series data is represented as a Fourier coefficient for that data, for example. In this case, the Fourier coefficient may be expressed in a complex form.

### Advantageous Effects of Invention

As is evident from the description above, with the blood vessel index value calculation apparatus according to an embodiment of the present invention, an index value indicating a state of a blood vessel can be calculated with greater accuracy than with prior techniques, based on information on a pulse wave acquired from the measurement subject.

Similarly, with the blood vessel index value calculation method according to an embodiment of the present invention, an index value indicating a state of a blood vessel can be calculated with greater accuracy than with prior techniques, based on information on a pulse wave acquired from the measurement subject.

Similarly, with the blood vessel index value calculation program according to an embodiment of the present invention, an index value indicating a state of a blood vessel can be calculated with greater accuracy than with prior techniques, based on information on a pulse wave acquired from the measurement subject.

### BRIEF DESCRIPTION OF DRAWINGS

FIG. 1 is a block diagram showing a configuration of a blood vessel index value calculation apparatus according to an embodiment of the present invention.

FIG. 2 is a block diagram showing a configuration of a first pulse wave sensor of the blood vessel index value calculation apparatus.

FIG. 3 is a block diagram showing a functional configuration of the blood vessel index value calculation apparatus.

FIG. 4 is a flowchart showing an overview of operations of the blood vessel index value calculation apparatus.

FIG. 5A shows an example of pulse wave time series data acquired at a right upper arm portion.

FIG. 5B shows an example of pulse wave time series data acquired at a left upper arm portion.

FIG. 5C shows an example of pulse wave time series data acquired at a right ankle portion.

FIG. 5D shows an example of pulse wave time series data acquired at a left ankle portion.

FIG. 6A is a graph showing frequency characteristics of data frames of the pulse wave time series data.

FIG. 6B is a graph showing frequency characteristics of the data frames of the pulse wave time series data resulting from noise removal processing.

FIG. 7A is a Bode diagram showing a frequency transfer characteristic of a vascular system (gain).

FIG. 7B is a Bode diagram showing a frequency transfer characteristic of a vascular system (phase).

FIG. 8 is a graph showing a distribution of a fundamental wave and harmonic waves of a pulse wave.

FIG. 9A is a Bode diagram showing a frequency transfer characteristic corrected by a frequency transfer characteristic smoothing unit (gain).

FIG. 9B is a Bode diagram showing a frequency transfer characteristic corrected by a frequency transfer characteristic smoothing unit (phase).

FIG. 10 is a Bode diagram showing a frequency transfer characteristic corrected by a frequency gain characteristic weighting unit (gain).

FIG. 11A is a Bode diagram showing a frequency transfer characteristic corrected by a frequency transfer characteristic band limiting unit (gain).

FIG. 11B is a Bode diagram showing a frequency transfer characteristic corrected by a frequency transfer characteristic band limiting unit (phase).

FIG. 12A is a Bode diagram showing a frequency transfer characteristic corrected by a frequency transfer characteristic low-band correction unit (gain).

FIG. 12B is a Bode diagram showing a frequency transfer characteristic corrected by a frequency transfer characteristic low-band correction unit (phase).

FIG. 13 is a graph showing a step response calculated by a step response calculation unit and an amount of time Tₚₑₐₖ that elapses before a peak, with the horizontal axis indicating sampling points.

FIG. 14A is a scatter diagram showing a relationship between ABWI values and ABI measurement values calculated by the ABWI calculation unit. (Using data in the case where noise removal processing is not performed by a noise-having block removal unit under predetermined conditions)

FIG. 14B is a scatter diagram showing a relationship between ABWI values and ABI measurement values. (Using data in the case where noise removal processing is furthermore performed by a noise-having block removal unit under the same conditions as in FIG. 14A)

FIG. 15A is a scatter diagram showing a relationship between ABWI values and ABI measurement values. (Using data in the case where frequency transfer characteristic correction processing is not performed by a frequency transfer characteristic smoothing unit under predetermined conditions)

FIG. 15B is a scatter diagram showing a relationship between ABWI values and ABI measurement values. (Using data in the case where frequency transfer characteristic correction processing is furthermore performed by a frequency transfer characteristic smoothing unit under the same conditions as in FIG. 15A)

FIG. 16A is a scatter diagram showing a relationship between ABWI values and ABI measurement values. (Using data in the case where frequency transfer characteristic correction processing is not performed by a frequency gain characteristic weighting unit under predetermined conditions)

FIG. 16B is a scatter diagram showing a relationship between ABWI values and ABI measurement values. (Using data in the case where frequency transfer characteristic correction processing is furthermore performed by a frequency gain characteristic weighting unit under the same conditions as in FIG. 16A)

FIG. 17A is a scatter diagram showing a relationship between ABWI values and ABI measurement values. (Using data in the case where frequency transfer characteristic correction processing is not performed by a frequency transfer characteristic band limiting unit under predetermined conditions)

FIG. 17B is a scatter diagram showing a relationship between ABWI values and ABI measurement values. (Using data in the case where frequency transfer characteristic correction processing is furthermore performed by a frequency transfer characteristic band limiting unit under the same conditions as in FIG. 17A)

FIG. 18A is a scatter diagram showing a relationship between ABWI values and ABI measurement values. (Using data in the case where ABWI values are calculated by an ABWI calculation unit without giving consideration to the pulse rate of the measurement subject under predetermined conditions)

FIG. 18B is a scatter diagram showing a relationship between ABWI values and ABI measurement values. (Using data in the case where ABWI values are calculated by an ABWI calculation unit with consideration given to the pulse rate of the measurement subject under the same conditions as in FIG. 18A)

### DESCRIPTION OF EMBODIMENTS

Hereinafter, an embodiment of the present invention will be described in detail with reference to the drawings.

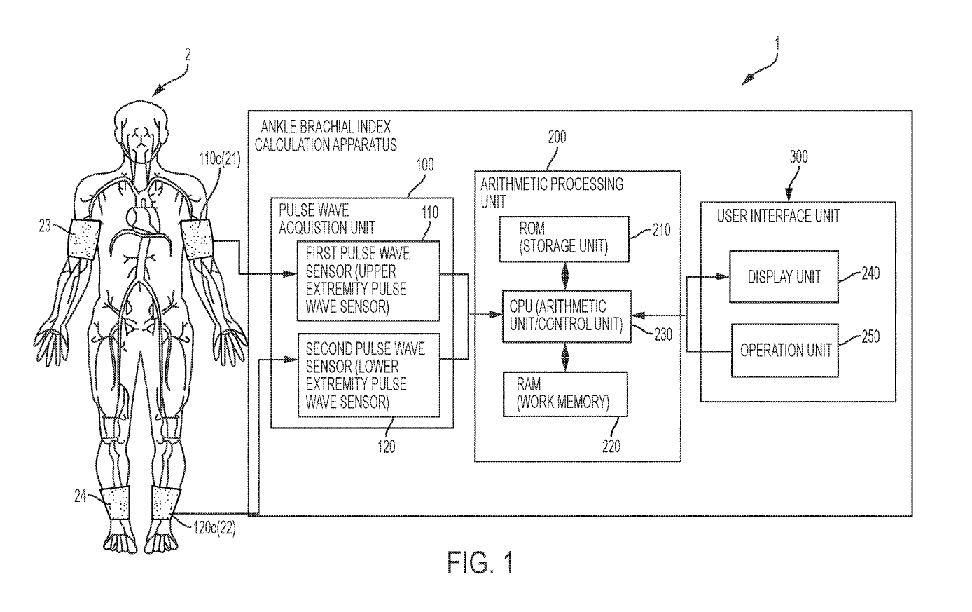

FIG. 1 is a block diagram showing a hardware configuration of an ankle brachial index calculation apparatus (indicated overall by reference numeral 1; abbreviated hereinafter as “ABI calculation apparatus”), which is a blood vessel index value calculation apparatus according to an embodiment of the present invention. The ABI calculation apparatus 1 has a pulse wave acquisition unit 100, an arithmetic processing unit 200, and a user interface unit 300, and can calculate an index value (e.g., an ABI value (hereinafter referred to as a “ABWI value”) calculated based on the pulse wave) indicating a state of a blood vessel of a measurement subject 2 based on data on a pulse wave acquired by the pulse wave acquisition unit 100. Here, ABWI is an abbreviation of Ankle Brachial Wave Index.

The pulse wave acquisition unit 100 measures a pulse wave of the measurement subject 2 and outputs the measurement result to the arithmetic processing unit 200. The pulse wave acquisition unit 100 has a first pulse wave sensor 110 that is connected to a first cuff 110c and can measure a pulse wave at a first measurement site (e.g., a left upper arm portion 21) of the measurement subject 2 and output the measurement result as time series data, and a second pulse wave sensor 120 that is connected to a second cuff 120c and can measure a pulse wave at a second measurement site (e.g., a left ankle portion 22) of the measurement subject 2 and output the measurement result as time series data. The first pulse wave sensor 110 and the second pulse wave sensor 120 may have substantially the same configuration, and both can independently measure a pulse wave of the measurement subject 2 synchronously due to control performed by the arithmetic processing unit 200.

Note that the pulse wave acquisition unit 100 may furthermore include third and fourth pulse wave sensors with configurations similar to those of the first and second pulse wave sensors 110 and 120, and may be able to measure pulse waves of the right upper arm portion 23 and the right ankle portion 24 of the measurement subject 2, for example, in synchronization with the first and second pulse wave sensors 110 and 120. Hereinafter, for the sake of simplicity in the description, a configuration for measuring pulse waves at two points, namely a first measurement site (left upper arm portion 21) included in the upper extremity of the measurement subject 2, and a second measurement site (left ankle portion 22) included in the lower extremity, is indicated at the pulse wave acquisition unit 100.

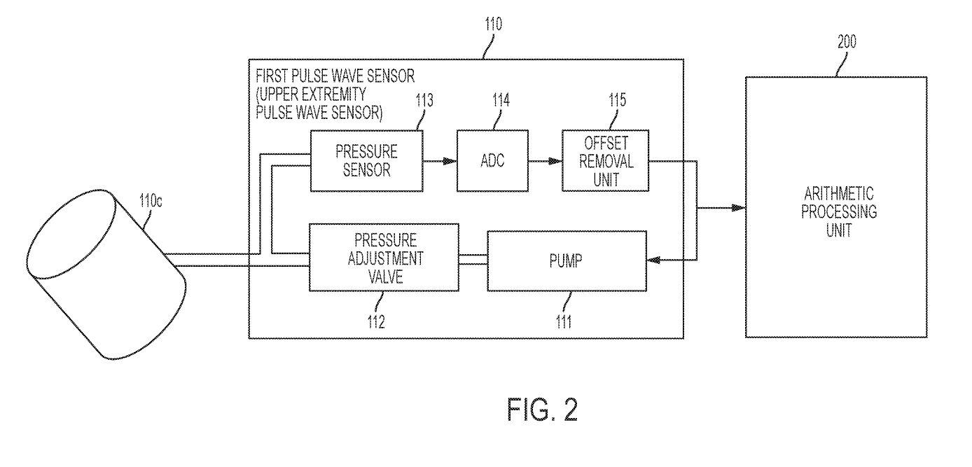

FIG. 2 is a diagram for describing details of a configuration of the pulse wave acquisition unit 100. For the sake of simplicity, the configuration of the second pulse wave sensor 120 is not included in the drawing, and only the configuration of the first pulse wave sensor 110 is shown. As described above, the configuration of the second pulse wave sensor 120 may be similar to that of the first pulse wave sensor 110.

The first pulse wave sensor 110 measures a pulse wave at a site at which the first cuff 110c is attached by adjusting the inner pressure of the first cuff 110c and performing detection. The first pulse wave sensor 110 includes a pump 111 that supplies air to the first cuff 110c, a pressure adjustment valve 112 for supplying and ejecting air to and from the first cuff 110c, a pressure sensor 113 that detects the pressure in the first cuff 110c, an analog-digital converter 114 (hereinafter referred to as “ADC”) that converts the output of the pressure sensor 113 into digital data, and an offset removal unit 115 that removes an offset component (a so-called DC component) from the output of the ADC 114 and outputs only a variation component (a so-called AC component).

When measuring the pulse wave, the first pulse wave sensor 110 is controlled by the arithmetic processing unit 200 to drive the pump 111 and retain the internal pressure of the first cuff 110c at about 50 mmHg, and the pressure sensor 113 detects the internal pressure of the first cuff 110c. The internal pressure detected by the pressure sensor 113 includes a pressure component that is maintained due to the action of the pump 111, and a pressure variation component caused by the pulse wave of the measurement subject 2. The ADC 114 converts the time series data on the pulse wave detected by the pressure sensor 113 into digital data at a predetermined rate [pts/sec] and the offset removal unit 115 removes the DC component from the digital data. In this manner, the time series data on the variation component of the pulse wave at the site at which the first cuff 110c is attached is output by the first pulse wave sensor 110 to the arithmetic processing unit 200.

Note that the pulse wave acquisition unit 100 is not limited to the configuration in which the pressure pulse wave is measured via the cuff as described above, and for example, it is possible to use a configuration in which the pulse wave is acquired optically.

Returning to FIG. 1, the arithmetic processing unit 200 includes a central processing unit 230 (hereinafter referred to as “CPU”) that performs processing for controlling arithmetic processing and the overall apparatus, a read-only memory 210 (hereinafter referred to as “ROM”) that stores programs to be executed by the CPU 230, and a random access memory 220 (hereinafter referred to as “RAM”) that is used as a work memory in various types of processing. Specifically, the ROM 210 stores a blood vessel index value calculation program for causing the control unit 230 to execute a blood vessel index value calculation method in which the ABI calculation apparatus 1 (blood vessel index value calculation apparatus) calculates an index value (e.g., an ABWI value) indicating the state of the blood vessel of the measurement subject 2, and the CPU 230 reads out the program stored in the ROM 210 and uses the RAM 220 to perform the following processing and calculate the blood vessel index value (e.g., the ABWI value).

The user interface unit 300 includes a display unit 240 and an operation unit 250. The display unit 240 includes a display screen (e.g., an LCD (Liquid Crystal Display), EL (Electroluminescence) display, or the like) and displays information relating to the pulse of the measurement subject 2 (e.g., the pulse rate), the ABWI value calculated by the ABI calculation apparatus 1, and the like. Control of the display screen is performed by the control unit 230 (CPU) (described later), which functions as a display control unit. The operation unit 250 includes a power supply switch that is operated in order to turn on or off the power supply of the ABI calculation apparatus 1, and a switch (start button) for starting calculation of the ABWI value. Note that the display unit 240 and the operation unit 250 may be constituted integrally using a touch panel type of display apparatus.

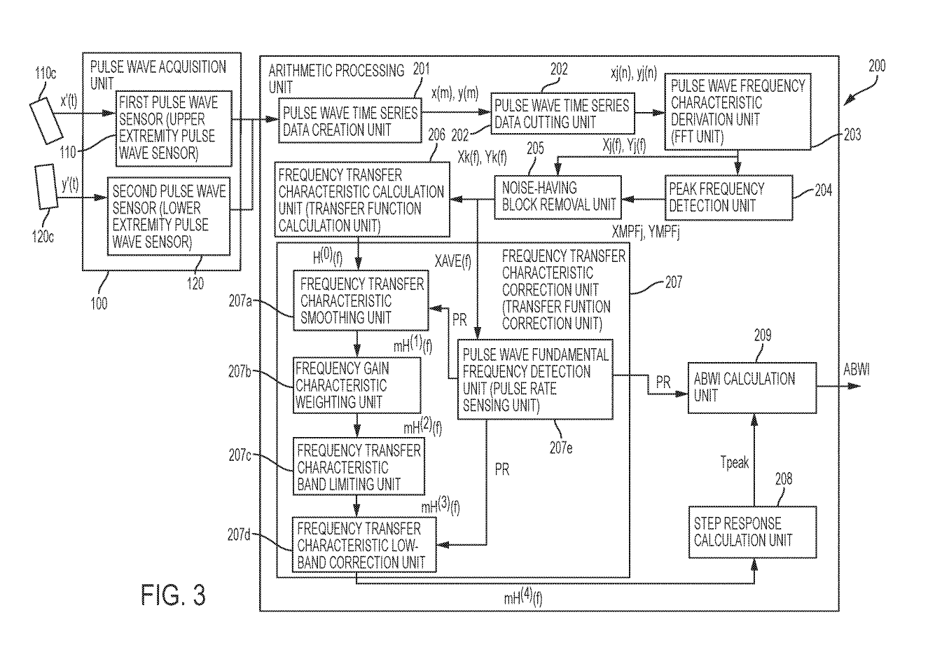

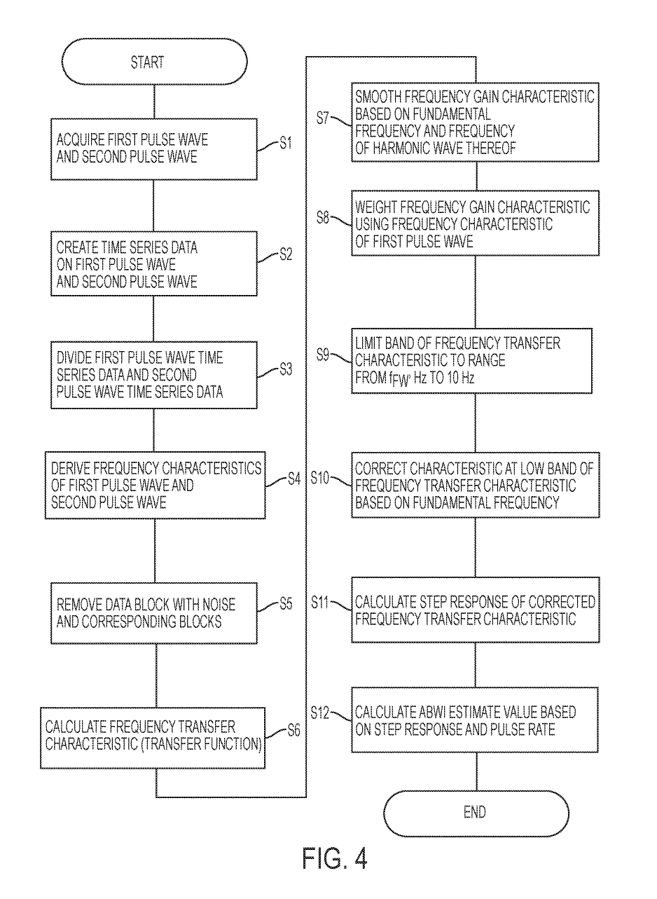

Next, functions realized by the arithmetic processing unit 200 of the ABI calculation apparatus 1, which is the blood vessel index value calculation apparatus, will be described with reference to FIGS. 3 to 13. FIG. 3 is a block diagram showing functions realized by the CPU 230 of the arithmetic processing unit 200 executing the above-described program. FIG. 4 is a flowchart showing an operation flow of the ABI calculation apparatus 1.

The pulse wave acquisition unit 100 measures the pulse wave at the first measurement site 21 of the measurement subject 2 for 30 seconds, and at the same time, measures the pulse wave at the second measurement site 22 for 30 seconds as well. Here, if x′(t) is the internal pressure of the first cuff 110c, y′(t) (t: 0 to 30 [seconds]) is the internal pressure of the second cuff 120c, the stable components of the internal pressures are x₀ and y₀ respectively, and the varying components of the internal pressures are x(t) and y(t) respectively,

x′(t)=x(t)+x₀ [Equation 1]

and

y′(t)=y(t)+y₀ [Equation 2]

are satisfied. The pulse wave acquisition unit 100 samples the varying components x(t) and y(t) of the pulse waves (first pulse wave and second pulse wave) of the first and second measurement sites 21 and 22 with a sampling frequency of 1200 [Hz] (1200 [pts/sec]) and outputs them as digital data to the arithmetic processing unit 200 (step S1 in FIG. 4).

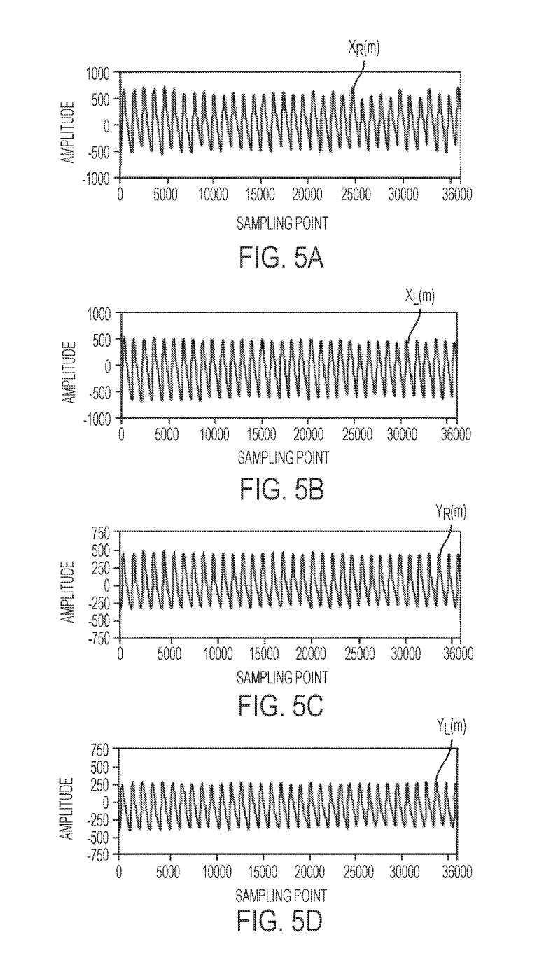

FIGS. 5A, 5B, 5C, and 5D show examples of digital data output by the pulse wave acquisition unit 100. FIG. 5A shows an example of pulse wave time series data acquired from the right upper arm portion 23, FIG. 5B shows an example of pulse wave time series data acquired from the left upper arm portion 21, FIG. 5C shows an example of pulse wave time series data acquired from the right ankle portion 24, and FIG. 5D shows an example of pulse wave time series data acquired from the left ankle portion 22. As described above, there is also a method of calculating the ABWI value using pulse waves at four points, namely the left and right upper arm portions and the left and right ankle portions, but here, in order to simplify the description, a method of calculating the ABWI value using two pieces of pulse wave time series data, namely the pulse wave at the left upper arm portion (data string x_{L}(m) in FIG. 5B) and the pulse wave at the left ankle portion (data string y_{L}(m) in FIG. 5D), will be described.

From the pulse wave acquisition unit 100, the pulse wave time series data creation unit 201 (CPU 230) of the arithmetic processing unit 200 acquires the digital data of the first and second pulse waves, which were obtained by sampling the first and second pulse waves with a sampling frequency of 1200 [Hz] for 30 seconds and removing the DC components therefrom (step S2 in FIG. 4). Hereinafter,

x(m) [Equation 3]

will be used as the time series data of the first pulse wave (times series data of the varying component), and

y(m) [Equation 4]

will be used as the time series data of the second pulse wave (times series data of the varying component). Here, m is an integer with a value of 1 to 36000.

A pulse wave time series data dividing unit 202 (CPU 230) receives the first and second pulse wave time series data x(m) and y(m) and divides them each into 16 data frames (blocks) with a frame size of 4096 data points and an overlapping ratio between adjacent data frames of 50% (2048 data points) (step S3 in FIG. 4). That is, the data of the j-th data frame (block) generated by dividing the first pulse wave time series data x(m) is in a relationship expressed by the following equation:

xⱼ(n)=x((j−1)×2048+n) [Equation 5]

and the data of the j-th data frame (block) generated by dividing the second pulse wave time series data y(m) is in a relationship expressed by the following equation:

yⱼ(n)=y((j−1)×2048+n) [Equation 6]

Here, j is an integer with a value of 1 to 16, and n is an integer with a value of 1 to 4096.

A pulse wave frequency characteristic derivation unit 203 (FFT unit) (CPU 230) converts the blocks xⱼ(n) of the first pulse wave time series data and the blocks yⱼ(n) of the second pulse wave time series data into frequency regions for each block (step S4 in FIG. 4). The conversion from the time regions of the blocks of the first and second pulse wave time series data into the frequency regions is performed by fast Fourier transform (FFT). Note that the processing for converting into the frequency regions may be performed using a method other than Fourier transform. Hereinafter, a complex-number Fourier coefficient obtained by converting a block of the j-th piece of first pulse wave time series data into a frequency region using FFT is

Xⱼ(f) [Equation 7]

and a complex-number Fourier coefficient obtained by converting a block of the j-th piece of second pulse wave time series data into a frequency region using FFT is

Yⱼ(f) [Equation 8]

Fourier coefficients Xⱼ(f) and Yⱼ(f) are such that

Xⱼ(f)=X_{jR}(f)+iX_{jI}(f)=X_{jA}(f)·e^{iX}^{_{jP}}^{(f)} [Equation 9]

and

Yⱼ(f)=Y_{jR}(f)+iY_{jI}(f)=Y_{jA}(f)·e^{iY}^{_{jP}}^{(f)} [Equation 10]

are satisfied. Here, X_{jR}(f) is the real number portion of Xⱼ(f), and X_{jI}(f) is the imaginary number portion of Xⱼ(f), and if polar coordinate notation in a complex plane is used, an amplitude X_{jA}(f) and a phase (argument) X_{jP}(f) are obtained. Similarly, Y_{jR}(f) is the real number portion of Yⱼ(f) and Y_{jI}(f) is the imaginary number portion of Yⱼ(f), and if polar coordinate notation in a complex plane is used, an amplitude Y_{jA}(f) and a phase (deflection angle) Y_{jP}(f) are obtained.

A peak frequency detection unit 204 (CPU 230) receives the Fourier coefficients Xⱼ(f) and Yⱼ(f) of the blocks of the first and second pulse wave data. The peak frequency detection unit 204 performs peak search (peak point detection) on the frequency amplitude characteristic of each block and transmits the lowest frequency of frequencies at which peaks were detected to a noise-having block removal portion 205 as the lowest peak frequency search result.

The noise-having block removal unit 205 (CPU 230) compares the lowest peak frequency search results for the blocks of the first pulse wave data, and based on the state of the distribution of the 16 lowest peak frequencies, estimates the lowest peak frequency indicated by the most search results as the fundamental frequency of the first pulse wave using decision by majority, for example. A block in which a frequency that is different from the estimated fundamental wave frequency is the lowest peak frequency is deemed as being a block in which noise is included, and is excluded (block rejection) from the processing following thereafter (step S5 in FIG. 4). A block of the second pulse wave data that corresponds to the excluded block of the first pulse wave data is also excluded from the processing following thereafter. The noise-having block removal unit 205 transmits only the frequency characteristics (Fourier coefficients) of the non-excluded blocks of the first and second pulse wave data to the frequency transfer characteristic calculation unit 206.

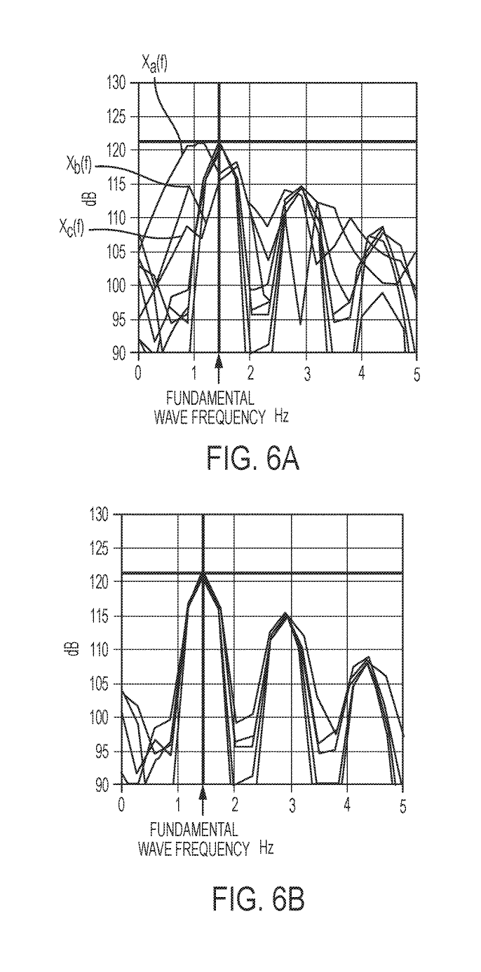

FIG. 6A is a graph in which the frequency amplitude characteristics of the blocks of the first pulse wave data before exclusion are plotted. As is evident from the drawing, the lowest peak frequency of the frequency amplitude characteristic Xₐ(f) of the a-th block, the lowest peak frequency of the frequency amplitude characteristic X_{b}(f) of the b-th block, and the lowest peak frequency of the frequency amplitude characteristic X_{c}(f) of the c-th block are significantly misaligned from the lowest peak frequency of the frequency amplitude characteristics of the other blocks. In this case, the noise-having block removal unit 205 deems the a-th block, the b-th block, and the c-th block as being blocks in which noise is included, and excludes them from the subsequent processing (block rejection).

FIG. 6B is a graph of the frequency amplitude characteristics of the non-excluded blocks. In this manner, the ABI calculation apparatus 1 according to the present embodiment obtains the lowest frequency exhibiting a peak in the frequency characteristic of each block, specifies and excludes data frames in which noise is included based on the obtained lowest frequencies, and derives the first frequency characteristic and the second frequency characteristic based on the non-excluded data frames of at least one of the first pulse wave data and the second pulse wave data and the corresponding data frames of at least the other of the first pulse wave data and the second pulse wave data. By doing so, the influence of noise that is added during pulse wave data acquisition is reduced, and the index value indicating the state of the blood vessel is calculated accurately.

Hereinafter, in order to simplify the description, Xk(f) and Yk(f) will be used as the frequency characteristics of the blocks of the first and second pulse wave data that were not excluded but were transmitted to the frequency transfer characteristic calculation unit 206. Here, k is an integer with a value of 1 to a block number K, which is the number of remaining blocks that were not excluded.

Note that in addition to or instead of the above-described processing, the noise-having block removal unit 205 may compare the lowest peak frequency search results for the blocks of the second pulse wave data, estimate the lowest peak frequency indicated by the most search results as the fundamental frequency of the second pulse wave based on the state of the distribution of the 16 lowest peak frequencies, deem a block in which a frequency that differs from the estimated fundamental frequency is the lowest peak frequency as being a block in which noise is included, and exclude it from subsequent processing (block rejection). In this case, a block of the first pulse wave data that corresponds to the excluded block of the second pulse wave data is also excluded from the subsequent processing.

The frequency transfer characteristic calculation unit 206 (transfer function calculation unit) (CPU 230) uses the blocks of the first and second pulse wave data that were not excluded by the noise-having block exclusion unit 205 to calculate a frequency transfer characteristic (so-called transfer function) of the vascular system in which the first pulse wave is used as the input and the second pulse wave is used as the output (step S6 in FIG. 4). The frequency transfer characteristic calculation unit 206 obtains a transfer function H⁽⁰⁾(f) using pairs (pair k (k: 1 to K)) of blocks of the first and second pulse wave data.

Specifically, the transfer function H⁽⁰⁾(f) is calculated using the following equation.

$${H}^{( 0 )} ( f ) = {G}^{( 0 )} ( f ) \cdot {ϕ}^{( 0 )} ( f ) = \frac{{Y}_{AVE} ( f ) \cdot {X}_{AVE}^{*} ( f )}{{X}_{AVE} ( f ) \cdot {X}_{AVE}^{*} ( f )} \qquad [ Equation 11 ]$$

Here, the index * indicates a complex conjugate, G⁽⁰⁾(f) indicates the frequency gain characteristic, and φ⁽⁰⁾(f) indicates the frequency phase characteristic. In the present embodiment, the equation above is used to derive the transfer function (frequency transfer characteristic) as the ratio between the cross spectrum of the input and output and the power spectrum of the input, but the equation above is merely an example of calculating the transfer function, and the transfer function may be calculated using an equation different from the equation above. X_{AVE}(f) and Y_{AVE}(f) are the averages of the frequency characteristics of the blocks that were not excluded by the noise-having block removal unit 205. For example, X_{AVE}(f) and Y_{AVE}(f) are such that

$$\begin{matrix}{X}_{AVE} ( f ) = \frac{1}{K} {\sum }_{k} {X}_{k} ( f ) & [ Equation 12 ] \\ {Y}_{AVE} ( f ) = \frac{1}{K} {\sum }_{k} {Y}_{k} ( f ) & [ Equation 13 ]\end{matrix}$$

are satisfied. Note that hereinafter, g will be used to indicate the frequency gain characteristic G in decibel notation, and θ (unit: radians) will be used as the phase of the frequency phase characteristic φ. That is,

g(f)[dB]=10 log G(f) [Equation 14]

φ(f)=e^{iθ(f)} [Equation 15]

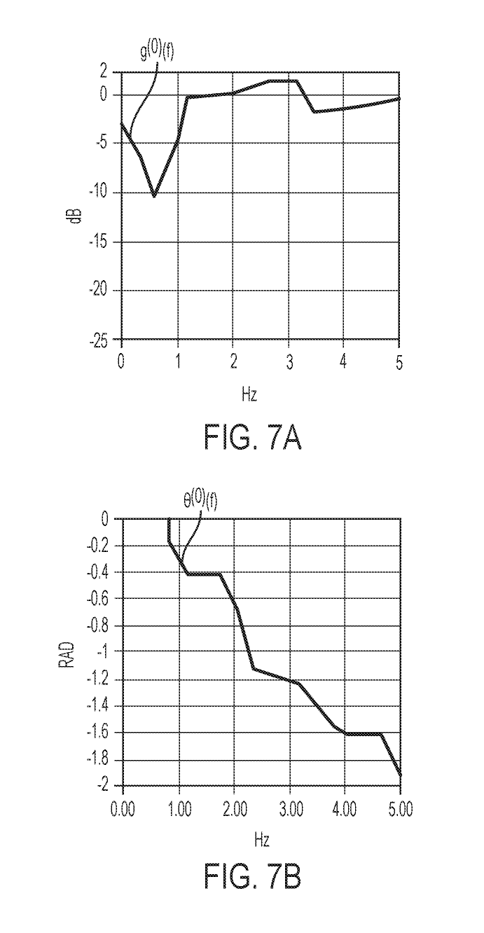

FIGS. 7A and 7B are Bode diagrams of the frequency transfer characteristic H⁽⁰⁾(f). FIG. 7A is a graph (unit: decibels) of the frequency gain characteristic of the frequency transfer characteristic H⁽⁰⁾(f), and FIG. 7B is a graph (unit: radians) of the frequency phase characteristic of the frequency transfer characteristic H⁽⁰⁾(f).

Next, the frequency transfer characteristic correction unit 207 (transfer function correction unit) (CPU 230) corrects the frequency transfer characteristic H⁽⁰⁾(f) calculated by the frequency transfer characteristic calculation unit 206 in the manner described below and outputs a corrected frequency transfer characteristic mH⁽⁴⁾(f).

A pulse wave fundamental frequency detection unit 207e (pulse rate sensing unit) (CPU 230) of the frequency transfer characteristic correction unit 207 receives the average frequency characteristic X_{AVE}(f) of the first pulse wave from the noise-having block removal unit 205, performs peak search on the frequency amplitude characteristic, and obtains the frequency f_{FW} of the fundamental wave included in the first pulse wave based on the frequency at which the peak was detected. In addition, the pulse wave fundamental frequency detection unit 207e determines the pulse rate PR of the measurement subject 2 based on the obtained frequency of the fundamental wave. The fundamental wave frequency f_{FW} (or pulse rate PR) obtained in this manner is transmitted to a frequency transfer characteristic smoothing unit 207a.

The frequency transfer characteristic smoothing unit 207a (CPU 230) of the frequency transfer characteristic correction unit 207 corrects the frequency gain characteristic G⁽⁰⁾(f) (i.e., g⁽⁰⁾(f)) and the frequency phase characteristic φ⁽⁰⁾(f) of the frequency transfer characteristic H⁽⁰⁾(f) based on the fundamental wave frequency f_{FW} transmitted by the pulse wave fundamental frequency detection unit 207e.

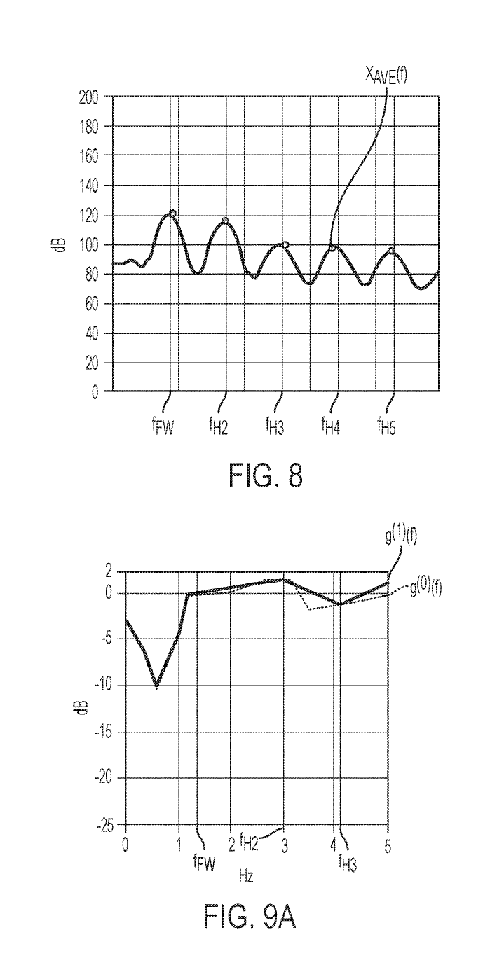

Specifically, the frequency transfer characteristic smoothing unit 207a first obtains the frequencies f_{H2}, f_{H3}, f_{H4}, fits, of high-order harmonic waves and the like by multiplying the fundamental wave frequency f_{FW} by an integer. FIG. 8 is a graph showing a relationship between the average frequency characteristic X_{AVE}(f), the fundamental wave frequency f_{FW}, the obtained frequencies f_{H2}, f_{H3}, f_{H4}, f_{H5} of the high-order harmonic waves, and the like.

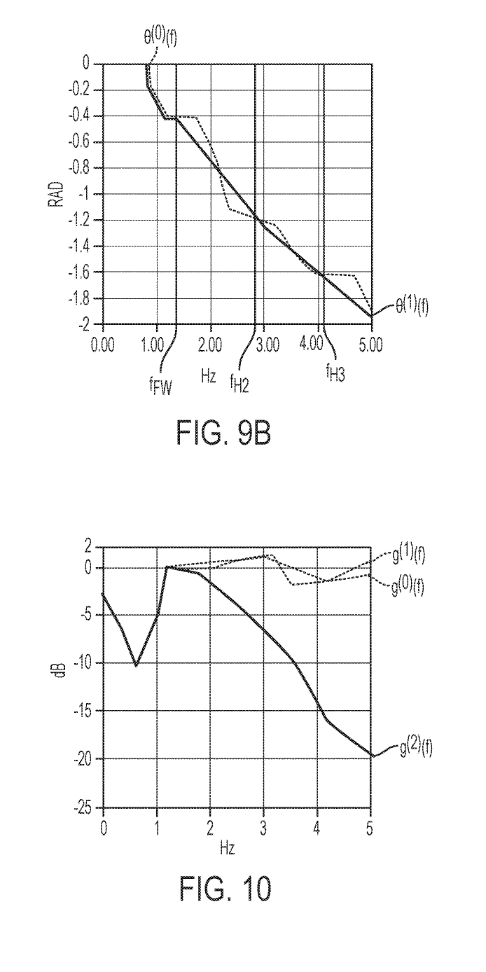

Also, the frequency transfer characteristic smoothing unit 207a uses the fundamental wave frequency f_{FW}, the frequencies f_{H2}, f_{H3}, f_{H4}, f_{H5} of the high-order harmonic waves, and the like to correct the frequency gain characteristic G⁽⁰⁾(f) (i.e., g⁽⁰⁾(f)) and the frequency phase characteristic φ⁽⁰⁾(f). FIG. 9A is a Bode diagram of the frequency gain characteristic g⁽⁰⁾(f) obtained due to the frequency gain characteristic g⁽⁰⁾(f) being corrected by the frequency transfer characteristic smoothing unit 207a. Hereinafter, a method for deriving the frequency gain characteristic g⁽¹⁾f) will be described. First, the frequency transfer characteristic smoothing unit 207a corrects the frequency gain characteristic G⁽⁰⁾(f) such that the gain G⁽⁰⁾(f_{FW}) at the fundamental wave frequency f_{FW} (first frequency) of the first pulse wave and the gain G⁽⁰⁾(f_{H2}) at the frequency f_{H2} (second frequency) of the second harmonic wave are connected by a straight line in the frequency gain characteristic G⁽⁰⁾(f).

Similarly, the frequency transfer characteristic smoothing unit 207a corrects the frequency gain characteristic G⁽⁰⁾(f) such that the gain G⁽⁰⁾(f_{H2}) at the frequency f_{H2} (second frequency) of the second harmonic wave of the first pulse wave and the gain G⁽⁰⁾(f_{H3}) at the frequency f_{H3} (third frequency) of the third harmonic wave are connected by a straight line in the frequency gain characteristic G⁽⁰⁾(f). In a similar manner thereafter, the frequency transfer characteristic smoothing unit 207a corrects the frequency gain characteristic G⁽⁰⁾(f) such that the k-th harmonic wave of the first pulse wave and the (k+1)-th harmonic wave are connected by a straight line for the third harmonic wave f_{H3} of the first pulse wave and the fourth harmonic wave f_{H4}, the fourth harmonic wave f_{H4} and the fifth harmonic wave f_{H5}, and so on in the frequency gain characteristic G⁽⁰⁾(f) (k being an integer with a value of 1 or more, and the first harmonic wave being the fundamental wave).

Next, the method for correcting the frequency phase characteristic φ⁽⁰⁾(f) will be described. FIG. 9B is a Bode diagram of the frequency phase characteristic φ⁽⁰⁾(f) obtained due to the frequency phase characteristic φ⁽⁰⁾(f) being corrected by the frequency transfer characteristic smoothing unit 207a. Similarly to the frequency gain characteristic g⁽⁰⁾(f), the frequency transfer characteristic smoothing unit 207a corrects the frequency phase characteristic φ⁽⁰⁾(f) such that the phase θ⁽⁰⁾(f_{FW}) at the frequency f_{FW} (first frequency) of the fundamental wave of the first pulse wave and the phase θ⁽⁰⁾(f_{H2}) at the frequency f_{H2} (second frequency) of the second harmonic wave are connected by a straight line, and the phase θ⁽⁰⁾(f_{H2}) of the frequency f_{H2} (second frequency) of the second harmonic wave and the phase θ⁽⁰⁾(f_{H3}) of the frequency f_{H3} (third frequency) of the third harmonic wave are connected by a straight line in the frequency phase characteristic φ⁽⁰⁾(f). In this manner, the frequency transfer characteristic smoothing unit 207a corrects the frequency phase characteristic φ⁽⁰⁾(f) such that the k-th harmonic wave of the first pulse wave and the (k+1)-th harmonic wave are connected by a straight line for the phase θ⁽⁰⁾(f) of the frequency phase characteristic φ⁽⁰⁾(f). Letting G⁽⁰⁾(f) and φ⁽⁰⁾(f) respectively be the frequency gain characteristic and the frequency phase characteristic corrected by the frequency transfer characteristic smoothing unit 207a in this manner, the corrected frequency transfer characteristic mH⁽¹⁾(f) is such that

mH⁽¹⁾(f)=G⁽¹⁾(f)·φ⁽¹⁾(f) [Equation 16]

is satisfied. Finally, the frequency transfer characteristic smoothing unit 207a transmits the frequency transfer characteristic mH⁽¹⁾(f) to a frequency gain characteristic weighting unit 207b (thus ends step S7 in FIG. 4).

The frequency transfer characteristic smoothing unit 207a corrects the frequency transfer characteristic H⁽⁰⁾(f) in the manner described above. By doing so, it is possible to suppress the contributions to the response to the later-described referential input of the components of the frequencies thought to not originate in the pulse wave in the frequency transfer characteristic, and it is possible for the later-described index value calculation unit to accurately calculate the index value (ABWI value) indicating the state of the blood vessel.

Next, the frequency gain characteristic weighting unit 207b (CPU 230) of the frequency transfer characteristic correction unit 207 receives the corrected frequency transfer characteristic mH⁽¹⁾(f) from the frequency transfer characteristic smoothing unit 207a, further corrects it, and outputs a corrected frequency transfer characteristic mH⁽²⁾(f).

Specifically, the frequency gain characteristic weighting unit 207b corrects the frequency gain characteristic by weighting the frequency gain characteristic G⁽¹⁾(f) of the corrected frequency transfer characteristic mH⁽¹⁾(f) based on the frequency amplitude characteristic X_{AVE}(f) of the first frequency characteristic and calculates the corrected frequency transfer characteristic mH⁽²⁾(f) based on the corrected frequency gain characteristic G⁽²⁾(f) (or g⁽²⁾(f)) and the frequency phase characteristic φ⁽¹⁾(f) (step S8 in FIG. 4). For example, the frequency transfer characteristic mH⁽²⁾(f) is such that

mH⁽²⁾(f)=G⁽²⁾(f)·φ⁽¹⁾(f) [Equation 17]

is satisfied. Here,

g⁽²⁾(f)[dB]10 log G⁽²⁾(f)=(10 log G⁽¹⁾(f))·|X_{AVE}(f)| [Equation 18]

is satisfied. FIG. 10 shows a plot of the corrected frequency gain characteristic g⁽²⁾(f). Thus, in the corrected frequency gain characteristic g⁽²⁾ (f), characteristics are reflected, the characteristics being such that the pulse wave includes the component of the frequency (frequency of fundamental wave) that coincides with the reciprocal of the pulse rate of the measurement subject and the components of the harmonic waves, and the amplitudes of the components decrease exponentially in the direction from the fundamental wave to the harmonic waves. By doing so, in the calculation of the later-described response, the response characteristic of the components of the fundamental wave and the comparatively low-order harmonic waves included in the pulse wave is emphasized in comparison to the response characteristic of the components of the comparatively high-order harmonic waves, and the later-described index value calculation unit can accurately calculate the index value indicating the state of the blood vessel based on the calculated response. Note that the specific method for weighting the frequency transfer characteristic G⁽²⁾(f) (or g⁽²⁾(f)) is not limited to the equation above. It is sufficient that, by performing weighting, the relative magnitude relationship between the amplitudes of the fundamental wave and the harmonic waves of the first pulse wave is reflected in the frequency transfer characteristic G⁽²⁾(f) (or g⁽²⁾(f)).

Next, a frequency transfer characteristic band limiting unit 207c (CPU 230) of the frequency transfer characteristic correction unit 207 receives the corrected frequency transfer characteristic mH⁽²⁾(f) from the frequency gain characteristic weighting unit 207b, further corrects it, and outputs a corrected frequency transfer characteristic mH⁽³⁾(f).

Specifically, the frequency transfer characteristic band limiting unit 207c obtains the further-corrected frequency transfer characteristic mH⁽³⁾(f) by limiting the frequency band of the corrected frequency transfer characteristic mH⁽²⁾(f) to a range between a frequency (low-band cut frequency f_{FW}′) (in units of Hertz) equal to a value obtained by subtracting at least the frequency resolution of the pulse wave frequency characteristic derivation unit 203 from the frequency f_{FW} of the fundamental wave of the first pulse wave, and 10 Hertz, and outputs the frequency transfer characteristic mH⁽³⁾(f) (step S9 in FIG. 4). It is sufficient that the low-band cut frequency f_{FW}′ is lower than the frequency f_{FW} of the fundamental wave of the first pulse wave (it is sufficient that it is less than the frequency f_{FW} of the fundamental wave of the first pulse wave). For example, if the fundamental wave frequency f_{FW} is 1.16 Hz and the frequency resolution of the pulse wave frequency characteristic derivation unit 203 is 0.29 Hz, the low-band cut frequency f_{FW}′ need only be set to no more than 1.16-0.29=0.87 Hertz.

That is, the frequency transfer characteristic mH⁽³⁾(f) is such that

mH⁽³⁾(f)=G⁽³⁾(f)·φ⁽³⁾(f) [Equation 19]

is satisfied. Here,

$$\begin{matrix}{G}^{( 3 )} ( f ) = \{ \begin{matrix}0 & ( f < {f}_{FW}^{′} [ Hz ] ) \\ {G}^{( 2 )} ( f ) & ( {f}_{FW}^{′} \leq f \leq 10 [ Hz ] ) \\ 0 & ( 10 < f [ Hz ] )\end{matrix} \\ and & [ Equation 20 ] \\ {ϕ}^{( 3 )} ( f ) = \{ \begin{matrix}0 & ( f < {f}_{FW}^{′} [ Hz ] ) \\ {ϕ}^{( 1 )} ( f ) & ( {f}_{FW}^{′} \leq f \leq 10 [ Hz ] ) \\ 0 & ( 10 < f [ Hz ] )\end{matrix} & [ Equation 21 ]\end{matrix}$$

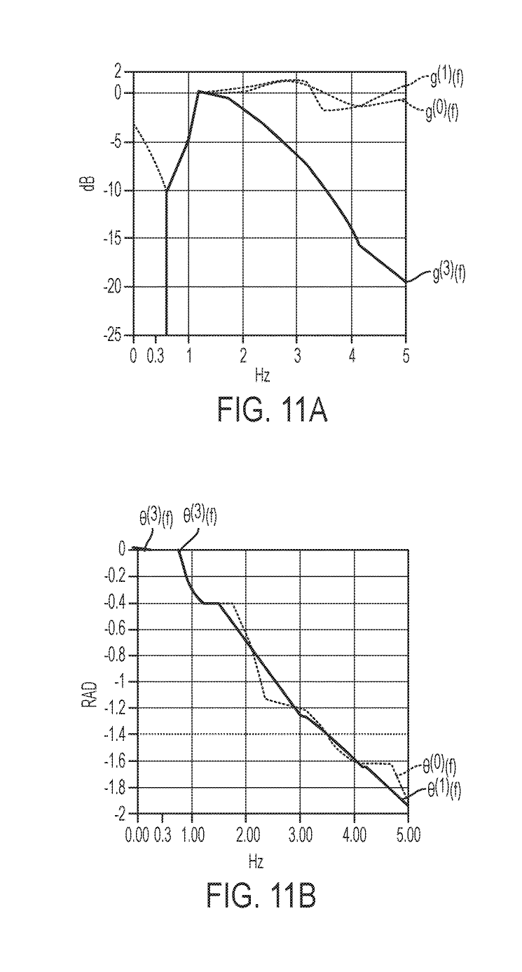

are satisfied. FIGS. 11A and 11B are Bode diagrams of the thus-obtained frequency gain characteristic g⁽³⁾(f) (=10 log(G⁽³⁾(f))) and the frequency phase characteristic φ⁽³⁾(f). In the examples shown in the drawings, the frequency band is limited to a range of 0.3 Hertz or more and 10 Hertz or less by setting the low-band cut frequency f_{FW}′ to 0.3 Hertz. The influence of the components of the comparatively high-order harmonic waves of the pulse wave on the response can be removed by limiting the frequency band of the frequency transfer characteristic to the low-band cut frequency f_{FW}′ Hertz or more and 10 Hertz or less in this manner. By doing so, the contributions from the components of the comparatively high-order harmonic waves of the pulse wave are removed (or at least reduced) in the response to the referential input, and the index value calculation unit can accurately calculate the index value by using such a response to calculate the index value (ABWI value) indicating the state of the blood vessel.

Next, a frequency transfer characteristic low-band correction unit 207c (CPU 230) of the frequency transfer characteristic correction unit 207 receives the corrected frequency transfer characteristic mH⁽³⁾(f) from the frequency transfer characteristic band limiting unit 207c, further corrects it, and outputs a corrected frequency transfer characteristic mH⁽⁴⁾(f).

Specifically, the frequency transfer characteristic low-band correction unit 207d corrects the frequency gain characteristic such that the frequency gain characteristic G⁽³⁾(f) in the frequency range of being less than or equal to the first frequency, which corresponds to the fundamental frequency f_{FW} of the first pulse wave, is constant at the gain G⁽³⁾(f_{FW}) at the first frequency, and this is used as the corrected frequency gain characteristic G⁽⁴⁾(f). Also, the frequency transfer characteristic low-band correction unit 207d corrects the frequency phase characteristic φ⁽³⁾(f) such that the phase θ⁽³⁾(f) in the frequency range of being less than or equal to the first frequency f_{FW} is constant at the phase θ⁽³⁾(f_{FW}) at the first frequency f_{FW}, and this is used as the corrected frequency phase characteristic φ⁽⁴⁾(f). Also, the frequency transfer characteristic low-band correction unit 207d obtains a further-corrected frequency transfer characteristic mH⁽⁴⁾(f) based on the corrected frequency gain characteristic G⁽⁴⁾(f) and the corrected frequency phase characteristic φ⁽⁴⁾(f) and outputs it (step S107 in FIG. 4).

That is, the frequency transfer characteristic mH⁽⁴⁾(f) is such that

mH⁽⁴⁾(f)=G⁽⁴⁾(f)·φ⁽⁴⁾(f) [Equation 22]

is satisfied. Here,

$$\begin{matrix}{G}^{( 4 )} ( f ) = \{ \begin{matrix}0 & ( f < 0.3 [ Hz ] ) \\ {G}^{( 3 )} ( {f}_{FW} ) & ( 0.3 \leq f \leq {f}_{FW} [ Hz ] ) \\ {G}^{( 3 )} ( f ) & ( {f}_{FW} < f \leq 10 [ Hz ] ) \\ 0 & ( 10 < f [ Hz ] )\end{matrix} \\ and & [ Equation 23 ] \\ {ϕ}^{( 4 )} ( f ) = \{ \begin{matrix}0 & ( f < 0.3 [ Hz ] ) \\ {ϕ}^{( 3 )} ( {f}_{FW} ) & ( 0.3 \leq f \leq {f}_{FW} [ Hz ] ) \\ {ϕ}^{( 3 )} ( f ) & ( {f}_{FW} < f \leq 10 [ Hz ] ) \\ 0 & ( 10 < f [ Hz ] )\end{matrix} & [ Equation 24 ]\end{matrix}$$

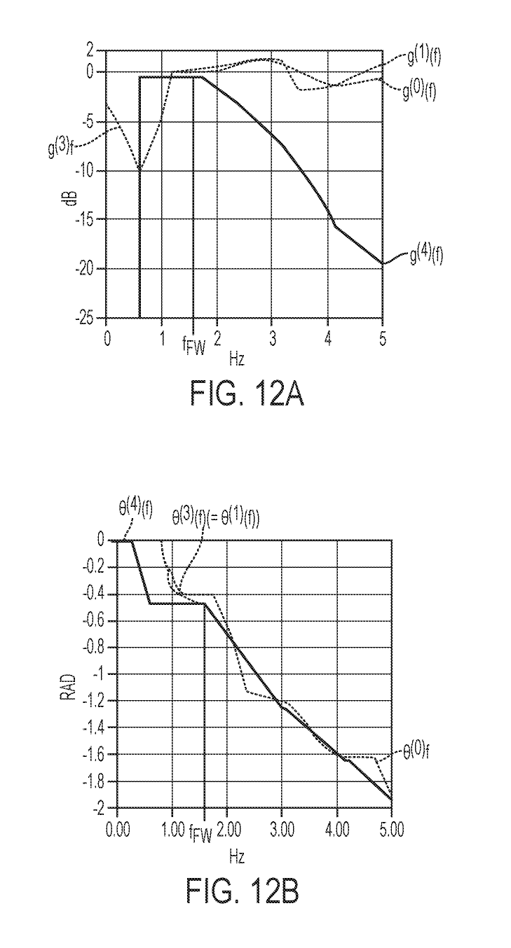

are satisfied. FIGS. 12A and 12B are Bode diagrams of the thus-obtained frequency gain characteristic g⁽⁴⁾(f) (=10 log(G⁽⁴⁾(f))) and the frequency phase characteristic φ⁽⁴⁾(f).

A step response calculation unit 208 (CPU 230) receives the corrected frequency transfer characteristic mH⁽⁴⁾(f) from the frequency transfer characteristic low-band correction unit 207d and calculates a response of the corrected frequency transfer characteristic mH⁽⁴⁾(f) to the referential input (e.g., a step function). Note that the referential input is not limited to being in the form of a step function.

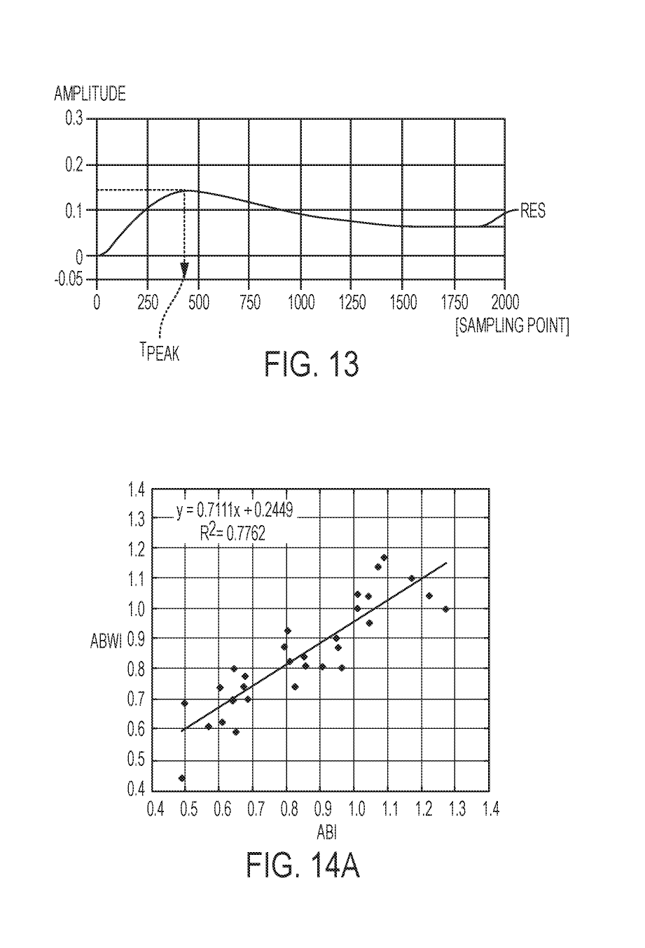

FIG. 13 is a plot of a response RES of the corrected frequency transfer characteristic mH⁽⁴⁾(f), which was obtained using a step function as the referential input. The horizontal axis in this case indicates the sampling point count. The zero-th point on the horizontal axis coincides with the time of inputting the referential input. The step response calculation unit 208 performs peak search on the response RES, specifies the first maximum that appears, and specifies the time (time on the time axis which is zero at the input time) Tₚₑₐₖ at which the maximum appears (step S11 in FIG. 4). The specified time Tₚₑₐₖ is transmitted to an ABWI calculation unit 209 serving as the index value calculation unit.

The ABWI calculation unit 209 (CPU 230) receives the time Tₚₑₐₖ from the step response calculation unit 208 and receives the pulse rate PR from the pulse wave fundamental frequency detection unit 207e. Then, the ABWI calculation unit 209 calculates the ABWI value based on the time Tₚₑₐₖ and the pulse rate PR (step S12 in FIG. 4).

The ABWI calculation unit 209 calculates the ABWI value (ABWI) using the following equation:

eABI=a·Tₚₑₐₖ b·PR+c [Equation 25]

Here, a, b, and c are coefficients obtained in advance. For example, the coefficients a, b, and c need only be obtained in advance by performing regression analysis using the time Tₚₑₐₖ and the pulse rate PR as independent variables and using the ABI value obtained by actually measuring the blood pressure as a dependent variable.

The thus-obtained ABWI value (ABWI) is transmitted to the display unit 240 and displayed on the display unit 240.

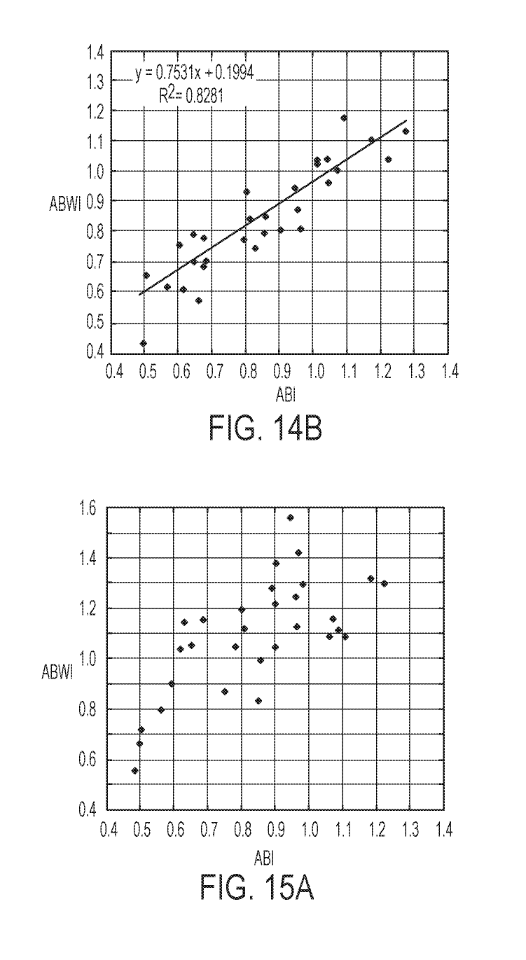

Hereinafter, the results of processing performed by the ABI calculation apparatus 1 of the present embodiment will be described with reference to FIGS. 14A to 18B.

FIGS. 14A and 14B show the results of a comparative test for indicating the effect of the processing performed by the noise-having block removal unit 205. FIG. 14A is a scatter diagram showing ABWI values (ABWI) and ABI values (ABI measurement values) obtained by measuring blood pressure, in the case of calculating the ABWI values (ABWI) under predetermined conditions without performing removal of the noise-having blocks using the noise-having block removal unit 205. FIG. 14B is a scatter diagram showing ABWI values (ABWI) and ABI measurement values in the case of performing removal of the noise-having blocks using the noise-having block removal unit 205 and calculating the ABWI values (ABWI) under the same conditions as in FIG. 14A. The other processing is the same in both cases. Note that the low-band cut frequency f_{FW}′ for band limiting performed by the frequency transfer characteristic band limiting unit 207c is set to a value obtained by subtracting the frequency value corresponding to the frequency resolution of the pulse wave frequency characteristic derivation unit 203 from the frequency of the fundamental wave of the first pulse wave. In other words, letting 0.29 Hertz be the frequency resolution of the pulse wave frequency characteristic derivation unit 203, the low-band cut frequency f_{FW}′ is obtained by solving f_{FW}′=f_{FW}−0.29, and this is used as the cut-off frequency on the low-band side in the band limiting.

From FIGS. 14A and 14B, it is understood that the correlation between the ABWI values and the ABI measurement values increases by performing the noise-having block removal processing using the noise-having block removal unit 205.

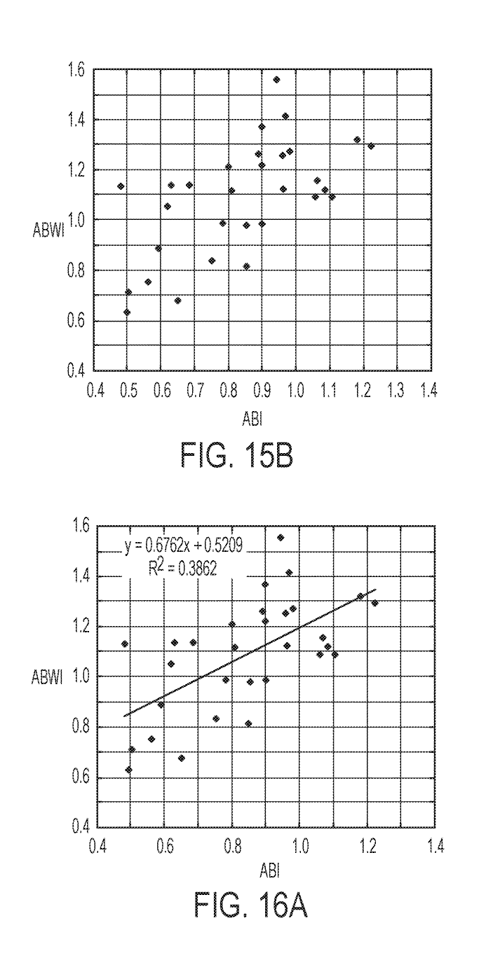

Next, FIGS. 15A and 15B show the results of a comparative test for indicating the effect of the processing performed by the frequency transfer characteristic smoothing unit 207a. FIG. 15A is a scatter diagram showing the ABWI values (ABWI) and ABI measurement values in the case of calculating the ABWI values (ABWI) under predetermined conditions without performing the frequency transfer characteristic smoothing processing performed by the frequency transfer characteristic smoothing unit 207a. FIG. 15B is a scatter diagram showing ABWI values (ABWI) and ABI measurement values in the case of performing frequency transfer characteristic smoothing processing using the frequency transfer characteristic smoothing unit 207a and calculating the ABWI values (ABWI) under the same conditions as in FIG. 15A. The other processing is the same in both cases. Note that the low-band cut frequency f_{FW}′ in the band limiting performed by the frequency transfer characteristic band limiting unit 207c is set to a value (f_{FW}′=f_{FW}−0.29) obtained by subtracting the frequency value corresponding to the frequency resolution of the pulse wave frequency characteristic derivation unit 203 from the frequency of the fundamental wave of the first pulse wave, similarly to the examples shown in FIGS. 14A and 14B.

From FIGS. 15A and 15B, it is understood that the correlation between the ABWI values and the ABI measurement values increases by performing the frequency transfer characteristic smoothing processing using the frequency transfer characteristic smoothing unit 207a.

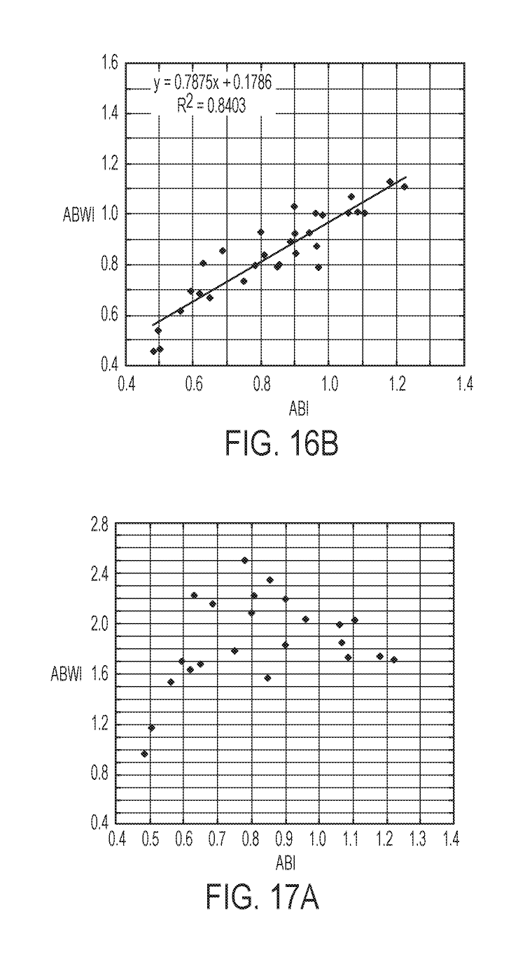

Next, FIGS. 16A and 16B show the results of a comparative test for indicating the effect of the processing performed by the frequency gain characteristic weighting unit 207b. FIG. 16A is a scatter diagram showing the ABWI values (ABWI) and ABI measurement values in the case of calculating the ABWI values (ABWI) under predetermined conditions without performing the weighting processing performed by the frequency gain characteristic weighting unit 207b. FIG. 16B is a scatter diagram showing ABWI values (ABWI) and ABI measurement values in the case of performing weighting processing using the frequency gain characteristic weighting unit 207b and calculating the ABWI values (ABWI) under the same conditions as in FIG. 16A. The other processing is the same in both cases. Note that the low-band cut frequency f_{FW}′ in the band limiting performed by the frequency transfer characteristic band limiting unit 207c is set to a value (f_{FW}′=f_{FW}−a0.29) obtained by subtracting the frequency value corresponding to the frequency resolution of the pulse wave frequency characteristic derivation unit 203 from the frequency of the fundamental wave of the first pulse wave, similarly to the examples shown in FIGS. 14A, 14B, 15A, and 15B.

From FIGS. 16A and 16B, it is understood that the correlation between the ABWI values and the ABI measurement values increases by performing the weighting processing using the frequency gain characteristic weighting unit 207b.

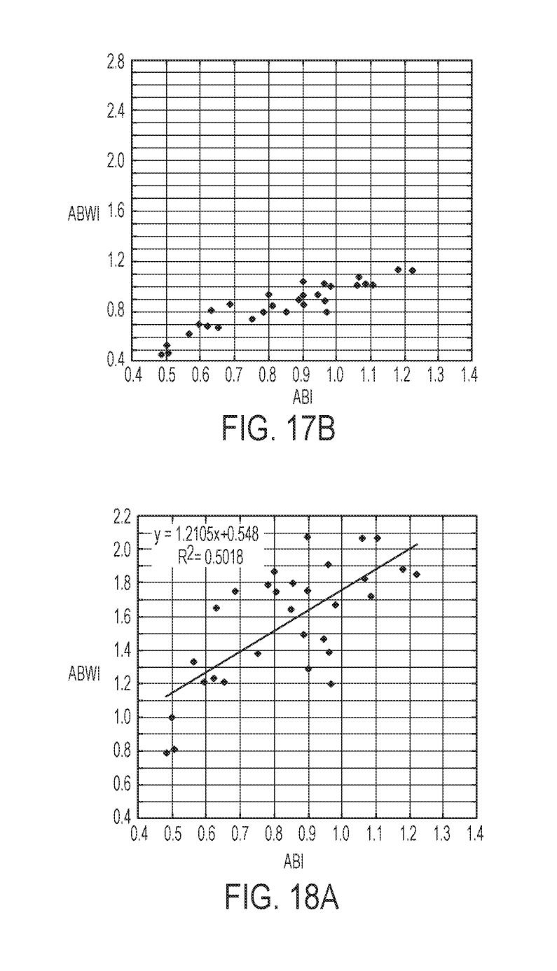

Next, FIGS. 17A and 17B show the results of a comparative test for indicating the effect of the processing performed by the frequency transfer characteristic band limiting unit 207c. FIG. 17A is a scatter diagram showing the ABWI values (ABWI) and ABI measurement values in the case of calculating the ABWI values (ABWI) under predetermined conditions without performing the band limiting processing performed by the frequency transfer characteristic band limiting unit 207c. FIG. 17B is a scatter diagram showing ABWI values (ABWI) and ABI measurement values in the case of performing band limiting processing using the frequency transfer characteristic band limiting unit 207c and calculating the ABWI values (ABWI) under the same conditions as in FIG. 17A. The other processing is the same in both cases. Note that the low-band cut frequency f_{FW}′ in the band limiting performed by the frequency transfer characteristic band limiting unit 207c in the derivation of the graph shown in FIG. 17B is set to a value (f_{FW}′=f_{FW}−0.29) obtained by subtracting the frequency value corresponding to the frequency resolution of the pulse wave frequency characteristic derivation unit 103 from the frequency of the fundamental wave of the first pulse wave, similarly to the examples shown in FIGS. 14A, 14B, 15A, 15B, 16A, and 16B.

From FIGS. 17A and 17B, it is understood that the correlation between the ABWI values and the ABI measurement values increases by performing the band limiting processing using the frequency transfer characteristic band limiting unit 207c.

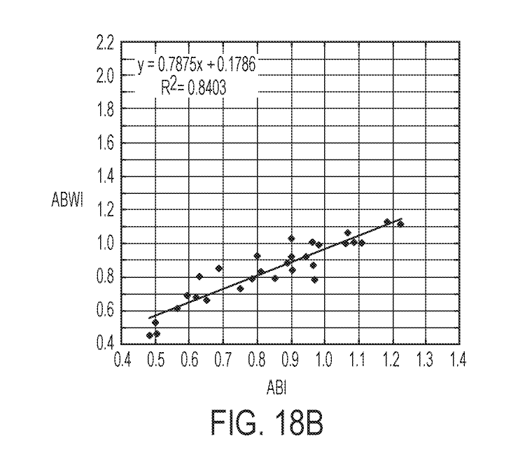

Finally, FIGS. 18A and 18B show the results of a comparative test for showing the effect of calculating the ABWI value with consideration given to the pulse rate PR using the ABWI calculation unit 209. FIG. 18A is a scatter diagram of ABWI values (ABWI) and ABI measurement values in the case of performing calculation using

eABI=a′·Tₚₑₐₖ+c′ [Equation 26]

without giving consideration to the pulse rate PR, under predetermined conditions. Here, a′ and c′ are, for example, coefficients obtained in advance by performing regression analysis using the time Tₚₑₐₖ as an independent variable and the ABI measurement value as a dependent variable. FIG. 18B is a scatter diagram showing the ABWI values (ABWI) and ABI measurement values in the case of calculating the ABWI values (ABWI) with consideration given to the pulse rate PR as described above, under the same conditions as in FIG. 18A. The other processing is the same in both cases. Note that the low-band cut frequency f_{FW}′ in the band limiting performed by the frequency transfer characteristic band limiting unit 207c is set to a value (f_{FW}′=f_{FW}−0.29) obtained by subtracting the frequency value corresponding to the frequency resolution of the pulse wave frequency characteristic derivation unit 203 from the frequency of the fundamental wave of the first pulse wave, similarly to the examples shown in FIGS. 14A, 14B, 15A, 15B, 16A, 16B, and 17B.

From FIGS. 18A and 18B, it is understood that the correlation between the ABWI values and the ABI measurement values increases due to consideration being given to the pulse rate PR when obtaining the ABWI values. The time Tₚₑₐₖ, which is an amount used when obtaining the ABWI values, is an amount having the dimension of time, which is considerably influenced by fluctuations in the pulse rate of the measurement subject. However, it is thought that the influence of these fluctuations can be reduced by giving consideration to the pulse rate PR of the measurement subject when obtaining the ABWI values based on the time Tₚₑₐₖ.

Note that the numerical values included in the above-described embodiments are all merely examples and may be changed as appropriate. Such changes are encompassed in the scope of the invention of this application.

The upper extremity, which is a site at which the first pulse wave is measured, includes an upper arm portion, a forearm portion, a hand, and the like.

If the pressure pulse wave measurement for the lower extremity is performed at sites such as an upper thigh portion, a lower thigh portion, a calf, a foot center, and a toe in addition to an ankle, it is possible to infer not only the existence of disease but also where the disease exists.

In the present embodiment, a cuff is used in order to measure the pulse wave of the measurement subject. However, the internal pressure of the cuff is retained at a comparatively low pressure such as 50 mmHg during pulse wave measurement. For this reason, the burden on the measurement subject can be reduced. This is because with patients of severe PAD, pain accompanies even slight compression of body parts. For this reason, blood pressure measurement, which requires an increase in cuff pressure to about 200 mmHg to 250 mmHg, is an examination that is difficult and painful for these patients. Blood vessels of patients who are afflicted with diabetes or are receiving dialysis are calcified in some cases, in which case the cuff pressure needs to be raised even higher than normal in order to measure the blood pressure, which results in even greater pain. Even so, it should be noted that arrhythmia and involuntary movement are sometimes observed in patients who are afflicted with diabetes or are receiving dialysis and PAD patients, and in such cases, it is difficult to obtain accurate blood pressure values. Even in such cases, it is possible to accurately calculate the ABI value using the ABI calculation apparatus 1 (blood vessel index value calculation apparatus) according to the present embodiment.

With the ABI calculation apparatus (blood vessel index value calculation apparatus) according to the present embodiment, there is no need to measure the blood pressure values, and an index corresponding to the ABI can be calculated in a shorter time than with a normal ABI examination. Accordingly, the present apparatus can reduce the burden conventionally felt by patients.

### REFERENCE SIGNS LIST

1 ABI calculation apparatus (blood vessel index value calculation apparatus)
100 Pulse wave acquisition unit
110 First pulse wave sensor
110c First cuff
120 Second pulse wave sensor
120c Second cuff
200 Arithmetic processing unit
210 ROM
220 RAM
230 CPU
300 User interface unit
240 Display unit
250 Operation unit

1. A blood vessel index value calculation apparatus for calculating an index value indicating a state of a blood vessel of a measurement subject, the blood vessel index value calculation apparatus comprising:
a pulse wave acquisition unit configured to acquire first pulse wave data that includes time series information on a first pulse wave, which is a pulse wave at a first measurement site of the measurement subject, and second pulse wave data that includes time series information on a second pulse wave, which is a pulse wave at a second measurement site of the measurement subject;
a pulse wave frequency characteristic derivation unit configured to derive a first frequency characteristic, which is a frequency characteristic of the first pulse wave, by converting the acquired first pulse wave data into a frequency space, and to derive a second frequency characteristic, which is a frequency characteristic of the second pulse wave, by converting the acquired second pulse wave data into a frequency space;
a frequency transfer characteristic calculation unit configured to, based on the first frequency characteristic and the second frequency characteristic, calculate a frequency transfer characteristic for a vascular system that includes the blood vessel and uses the first pulse wave as input and the second pulse wave as output;
a frequency transfer characteristic correction unit configured to correct the calculated frequency transfer characteristic;
a response calculation unit configured to calculate a response of the vascular system to pre-determined referential input using the corrected frequency transfer characteristic;
an index value calculation unit configured to calculate an index value indicating the state of the blood vessel based on the calculated response, and
wherein the frequency transfer characteristic correction unit corrects a frequency gain characteristic of the frequency transfer characteristic by weighting the frequency gain characteristic based on a frequency amplitude characteristic of the first frequency characteristic, and corrects the frequency transfer characteristic based on the corrected frequency gain characteristic.

2. The blood vessel index value calculation apparatus according to claim 1, wherein based on the first frequency characteristic, the frequency transfer characteristic correction unit corrects the frequency gain characteristic of the frequency transfer characteristic such that frequency gain characteristics of a first frequency, which is equivalent to a frequency of a fundamental wave of the frequency transfer characteristic, and a second frequency, which is equivalent to a frequency of a second harmonic wave of the first frequency, pass through gains at the first frequency and the second frequency and change linearly,
corrects a frequency phase characteristic of the frequency transfer characteristic such that frequency phase characteristics of the first frequency and the second frequency pass through phases at the first frequency and the second frequency and change linearly, and
corrects the frequency transfer characteristic based on the corrected frequency gain characteristic and the corrected frequency phase characteristic.

3. The blood vessel index value calculation apparatus according to claim 2, wherein based on the first frequency characteristic, the frequency transfer characteristic correction unit corrects the frequency gain characteristic of the frequency transfer characteristic such that the frequency gain characteristics of the second frequency and a third frequency, which is equivalent to a frequency of a third harmonic wave of the first pulse wave, pass through gains at the second frequency and the third frequency and change linearly,
corrects the frequency phase characteristic of the frequency transfer characteristic such that the frequency phase characteristics of the second frequency and the third frequency pass through phases at the second frequency and the third frequency and change linearly, and
corrects the frequency transfer characteristic based on the corrected frequency gain characteristic and the corrected frequency phase characteristic.

4. The blood vessel index value calculation apparatus according to claim 1, wherein
the frequency transfer characteristic correction unit corrects the frequency transfer characteristic by restricting a frequency band of the frequency transfer characteristic to be within a range from a frequency lower than the frequency of the fundamental wave of the first pulse wave to 10 Hertz.

5. The blood vessel index value calculation apparatus according to claim 1, wherein the referential input is in the form of a step function, the response calculation unit calculates the response of the vascular system to the referential input, and
the index value calculation unit calculates the index value indicating the state of the blood vessel based on an amount of time that elapses before a maximum first appears in the response.

6. The blood vessel index value calculation apparatus according to claim 1, wherein based on the first frequency characteristic,
the frequency transfer characteristic correction unit corrects the frequency phase characteristic of the frequency transfer characteristic such that the frequency phase characteristic in a frequency range of being less than or equal to the first frequency reaches a value equal to the phase at the first frequency, and
corrects the frequency transfer characteristic based on the corrected frequency phase characteristic.

7. The blood vessel index value calculation apparatus according to claim 1, wherein based on the first frequency characteristic,
the frequency transfer characteristic correction unit corrects the frequency gain characteristic of the frequency transfer characteristic such that the frequency gain characteristic in a frequency range of being less than or equal to the first frequency, which is equivalent to the frequency of the fundamental wave of the first pulse wave, reaches a value equal to the gain at the first frequency, and
corrects the frequency transfer characteristic based on the corrected frequency gain characteristic.

8. The blood vessel index value calculation apparatus according to claim 1, wherein the index value calculation unit calculates the index value indicating the state of the blood vessel based on the response and the frequency of the fundamental wave of the first pulse wave.

9. The blood vessel index value calculation apparatus according to claim 1, wherein the pulse wave frequency characteristic derivation unit divides the first pulse wave data and the second pulse wave data into a plurality of data frames, derives frequency characteristics of the data frames of at least one of the first pulse wave data and the second pulse wave data, obtains the lowest frequency exhibiting a peak in each derived frequency characteristic, specifies and excludes a data frame including noise based on the obtained lowest frequency, and derives the first frequency characteristic and the second frequency characteristic based on the data frames of at least one of the first pulse wave data and the second pulse wave data that were not excluded, and on the corresponding data frames of at least the other of the first pulse wave data and the second pulse wave data.

10. A blood vessel index value calculation method for calculating an index value indicating a state of a blood vessel of a measurement subject using a blood vessel index value calculation apparatus, the method comprising:
a step in which an arithmetic unit of the blood vessel index value calculation apparatus acquires first pulse wave data that includes time series information on a first pulse wave, which is a pulse wave at a first measurement site of the measurement subject, and second pulse wave data that includes time series information on a second pulse wave, which is a pulse wave at a second measurement site of the measurement subject;
a step in which the arithmetic unit derives a first frequency characteristic, which is a frequency characteristic of the first pulse wave, by converting the acquired first pulse wave data into a frequency space, and derives a second frequency characteristic, which is a frequency characteristic of the second pulse wave, by converting the acquired second pulse wave data into a frequency space;
a step in which the arithmetic unit, based on the first frequency characteristic and the second frequency characteristic, calculates a frequency transfer characteristic for a vascular system that includes the blood vessel and uses the first pulse wave as input and the second pulse wave as output;
a step in which the arithmetic unit corrects the frequency transfer characteristic calculated in the step of calculation;
a step in which the arithmetic unit calculates a response of the blood vessel system to pre-determined referential input using the frequency transfer characteristic corrected in the step of correction;
a step in which the arithmetic unit calculates an index value indicating the state of the blood vessel based on the response calculated in the step of calculating the response, and
wherein the step of correction includes a step in which the arithmetic unit corrects a frequency gain characteristic of the frequency transfer characteristic by weighting the frequency gain characteristic based on a frequency amplitude characteristic of the first frequency characteristic, and corrects the frequency transfer characteristic based on the corrected frequency gain characteristic.

11. A blood vessel index value calculation program for causing a computer to execute a method for calculating an index value indicating a state of a blood vessel of a measurement subject, the method comprising:
a step of acquiring first pulse wave data that includes time series information on a first pulse wave, which is a pulse wave at a first measurement site of the measurement subject, and second pulse wave data that includes time series information on a second pulse wave, which is a pulse wave at a second measurement site of the measurement subject;
a step of deriving a first frequency characteristic, which is a frequency characteristic of the first pulse wave, based on the acquired first pulse wave data, and deriving a second frequency characteristic, which is a frequency characteristic of the second pulse wave, based on the acquired second pulse wave data;
a step of, based on the first frequency characteristic and the second frequency characteristic, calculating a frequency transfer characteristic for a vascular system that includes the blood vessel and uses the first pulse wave as input and the second pulse wave as output;
a step of correcting the frequency transfer characteristic calculated in the step of calculation;
a step of calculating a response of the blood vessel system to pre-determined referential input using the frequency transfer characteristic corrected in the step of correction; and
a step of calculating an index value indicating the state of the blood vessel based on the response calculated in the step of calculating the response,
wherein the step of correction includes a step of correcting a frequency gain characteristic of the frequency transfer characteristic by weighting the frequency gain characteristic based on a frequency amplitude characteristic of the first frequency characteristic, and correcting the frequency transfer characteristic based on the corrected frequency gain characteristic.
